(19) United States
(12) Patent Application Publication
(10) Pub. No.: US 2015/0125462 A1
(43) Pub. Date: May 7, 2015

(54) METHODS OF TREATING ANKYLOSING SPONDYLITIS USING IL-17 ANTAGONISTS

(71) Applicants: Stephan Bek, Basel (CH); Arndt Brachat, Lorrach (DE); Wolfgang Hueber, Basel (CH); Yali Li, Malden, MA (US); Ying Wang, Waltham, MA (US); Roman Yelensky, Newton, MA (US)

(72) Inventors: Stephan Bek, Basel (CH); Arndt Brachat, Lorrach (DE); Wolfgang Hueber, Basel (CH); Yali Li, Malden, MA (US); Ying Wang, Waltham, MA (US); Roman Yelensky, Newton, MA (US)

(21) Appl. No.: 14/394,617

(22) PCT Filed: Apr. 18, 2013
(86) PCT No.: PCT/US2013/037068

§ 371 Date: Oct. 15, 2014

Related U.S. Application Data
(60) Provisional application No. 61/636,062, filed on Apr. 20, 2012.

(51) Int. Cl. C07K 16/24; C12Q 1/68

(52) U.S. Cl. CPC C07K 16/244; C12Q 1/6883; C07K 2317/21; C07K 2317/76; A61K 2039/505
USPC 424/142.1; 435/6.11

(57) ABSTRACT

The disclosure is directed to novel personalized therapies and methods for treating ankylosing spondylitis (AS). Specifically, this disclosure relates to methods of treating a patient having AS by selectively administering an IL-17 antagonist, e.g., an IL-17 antibody, such as secukinumab, to the AS patient on the basis of that patient being predisposed to have a favorable response to treatment with the IL-17 antagonist. Also disclosed herein are diagnostic methods and transmittable forms of information useful in predicting the likelihood that a patient having AS will respond to treatment with an IL-17 antagonist, e.g., an IL-17 antibody, such as secukinumab.

This disclosure claims priority to U.S. Provisional Patent Application No. 61/636,062, filed Apr. 20, 2012, the disclosure of which is incorporated by reference herein in its entirety.

### TECHNICAL FIELD

The disclosure is directed to novel personalized therapies, kits, transmittable forms of information and methods for use in treating patients having ankylosing spondylitis (AS).

### BACKGROUND OF THE DISCLOSURE

Ankylosing spondylitis (AS) is a chronic inflammatory disease, which is mainly characterized by involvement of axial joints and bilateral sacroiliitis. Peripheral joints and extra-articular organs may also be involved in AS. Associated extra-articular manifestations include cardiovascular and pulmonary abnormalities, neurologic sequelae, and both clinical and subclinical gastrointestinal findings. Decreased bone mineral density (BMD) is typical of extra-articular symptoms and many patients with AS have osteoporosis and consequent non-traumatic fractures in spite of their young age and gender (male). Generalized osteoporosis as well as regional osteopenia is common in AS with high incidence of osteoporosis or osteopenia, in spine (41-62%) and in femur (46-86%). The presence of the HLA-B27 antigen is strongly associated with AS: 90-95% of patients with AS who have European ancestry carry this marker. AS affects up to 0.9% of the population and is associated with significant morbidity and disability, and thus constitutes a major socioeconomic burden.

The first-line drug treatments of mild AS are non-steroidal anti-inflammatory drugs (NSAIDs). Treatment of NSAIDs-refractory AS is hampered by the lack of efficacy of virtually all standard disease modifying anti-rheumatic drugs (DMARDs), including methotrexate (MTX). As an exception, peripheral arthritis associated with AS responds quite well to sulfasalazine. Tumor necrosis factor (TNF) blocking agents have been successfully used to treat AS (Braun J et al (2002) Lancet 359:1187-93) and demonstrate prolonged efficacy up to three years of follow-up (Braun et al. (2005) Rheumatology 44:670-6). However, upon discontinuation of TNF blockers, AS relapses quickly, indicating that the inflammatory process may only be suppressed by TNF blockade (Baraliakos et al (2005) Arthritis Rheum 53:856-63). Moreover, concerns exist about the short and long-term tolerability and safety of chronic TNF-alpha antagonisim in general, most notably the reactivation of serious infections (e.g., tuberculosis infections), liver toxicity, increased cardiovascular disease, induction (or exacerbation of) demyelinating conditions, and increased incidence of malignancy due to TNF-alpha antagonisim (M. Khraishi (2009) J. Rheumatol Suppl. 82:25-32; Salliot et al. (2009) Ann. Rheum. Dis. 68:25-32).

Secukinumab (AIN457) is a high-affinity fully human monoclonal anti-human antibody that inhibits Interleukin-17A (IL-17) activity. In a recent AS proof-of-concept (PoC) study (AIN457A2209), secukinumab has emerged as a potential treatment for patients with AS. However, since patient response to biological treatment is variable and it is desirable to avoid providing drug to patients who will be resistant thereto, there is a need to develop methods of treating AS that first identity those patients most likely to benefit from a chosen biological treatment.

### BRIEF SUMMARY OF THE DISCLOSURE

While several single nucleotide polymorphisms (SNPs) are linked to the AS disease state (Wellcome Trust et al (2007) Nat Genet. 39(11):1329-37), thus far no biomarker has been identified as being predictive of whether an AS patient will respond to a particular drug, e.g., an IL-17 antagonist. Provided herein are novel personalized therapies and methods for treating AS that maximize the benefit and minimize the risk of IL-17 antagonism in the AS population by first identifying those patients most likely to respond favorably to antagonism of IL-17 during treatment of AS. This finding is based, in part, on the determinations that:

1) the ERAP1 (endoplasmatic reticulum aminopeptidase 1) rs30187 “T” allele and the ERAP1 rs27434 “A” alleles (both of which are referred to herein as “AS non-response alleles”) associate with reduced ASAS (Assessment in SpondyloArthritis) 40 response during secukinumab treatment;

2) following secukinumab treatment, patients having at least one IL23R (Interleukin-23 receptor) rs11209032 “G” allele (referred to herein as an “AS response allele”) display improved Bath Ankylosing Spondylitis Disease Activity Index (BASDAI) scores over time relative to patients having only the rs11209032 “A” allele, and patients having at least one rs2201841 “T” allele (also referred to herein as an “AS response allele”) display improved BASDAI scores over time relative to patients having only the rs2201841 “C” allele;

3) elevated baseline serum levels of S100A8, S100A9 and S100A8/S100A9 (referred to herein as “AS response proteins”) associate with increased ASAS20 and ASAS40 response during secukinumab treatment; and

4) AS patients that carry either the ERAP1 risk alleles rs30187 (“T”) or rs27434 (“A”) typically show higher levels of ERAP1 expression, while homozygous noncarriers mostly show lower ERAP1 transcript levels.

We thus contemplate that testing subjects for the presence of one or more of these AS non-response alleles, AS response alleles and/or AS response proteins, or the level of ERAP1 expression, level of ERAP1 protein or level of ERAP1 activity will be useful in a variety of diagnostic products and methods that involve identifying individuals more likely to respond to IL-17 antagonsim and in helping physicians decide whether to prescribe IL-17 antagonists (e.g., secukinumab) to a patient having AS. Accordingly, it is one object of the disclosure to provide methods of treating AS, by selectively administering the patient a therapeutically effective amount of an IL-17 antagonist, e.g., an IL-17 antibody, such as secukinumab, based on certain aspects of the patient's biochemical profile. It is another object of the disclosure to provide methods of identifying patients who are more likely to respond to treatment of AS with an IL-17 antagonist, e.g., an IL-17 antibody, such as the AINI457 antibody (secukinumab) based on certain aspects of the patient's biochemical profile. It is another object of the disclosure to provide methods of determining the likelihood that an AS patient will respond to treatment with an IL-17 antagonist, e.g., an IL-17 antibody, such as secukinumab, based on certain aspects of the patient's biochemical profile.

Based upon the above objects and discoveries, disclosed herein are various methods of selectively treating a patient having AS. In some embodiments, these methods comprise assaying a biological sample from the patient for the presence (or absence) of an AS non-response allele, an AS response allele and/or an increased level of an AS response protein; and thereafter selectively administering a therapeutically effective amount of an IL-17 antagonist, e.g., secukinumab, to the patient if the patient does not have the AS non-response allele or if the patient has an AS response allele or if the patient has an increased level of an AS response protein. In other embodiments, these methods comprise assaying a biological sample from the patient for the level of ERAP1 expression (e.g., mRNA, cDNA, etc.), the level of ERAP1 protein, and/or the level of ERAP1 activity; and thereafter selectively administering a therapeutically effective amount of an IL-17 antagonist, e.g., secukinumab, to the patient if the patient has a decreased level of ERAP1 expression, decreased level of ERAP1 protein, and/or decreased level of ERAP1 activity relative to a control.

Disclosed herein are also various methods of predicting the likelihood that a patient having AS will respond to treatment with an IL-17 antagonist, e.g., secukinumab. In some embodiments, these methods comprise detecting the presence of an AS non-response allele in a biological sample from the patient, wherein the presence of the AS non-response allele is indicative of a decreased likelihood that the patient will respond to treatment with the IL-17 antagonist. In some embodiments, these methods comprise detecting the presence of an AS response allele in a biological sample from the patient, wherein the presence of the AS response allele is indicative of an increased likelihood that the patient will respond to treatment with the IL-17 antagonist. In some embodiments, these methods comprise detecting the level of an AS response protein in a biological sample from the patient, wherein an increase in the level of an AS response protein in the patient relative to a control level of the AS response protein is indicative of an increased likelihood that the patient will respond to treatment with the IL-17 antagonist. In other embodiments, these methods comprise detecting the level of ERAP1 expression (e.g., mRNA, cDNA, etc.), the level of ERAP1 protein, and/or the level of ERAP1 activity in a biological sample from the patient; wherein a decreased level of ERAP1 expression, decreased level of ERAP1 protein, and/or decreased level of ERAP1 relative to a control is indicative of an increased likelihood that the patient will respond to treatment with the IL-17 antagonist. In some of these embodiments, the step of detecting is performed by assaying the biological sample from the patient for the subject matter of interest.

In some embodiments, the IL-17 antagonist is an IL-17 binding molecule, preferably a human antibody, most preferably secukinumab.

Additional methods, uses, and kits are provided in the following description and appended claims. Further features, advantages and aspects of the present disclosure will become apparent to those skilled in the art from the following description and appended claims.

### BRIEF DESCRIPTION OF THE DRAWINGS

FIG. 1 shows the CAIN457A2209 clinical trial design. Patients received two infusions of 10 mg/kg secukinumab or placebo at days 1 and 22.

FIG. 2 shows the change of ASAS response rates for carriers and non-carriers of the ERAP1 SNP rs30187 non-response allele.

FIG. 3 shows the change of delta BASDAI scores (relative to baseline) for patients with different IL23R SNP rs11209032 genotypes.

FIG. 4 shows the change of delta BASDAI scores (relative to baseline) for patients with different IL23R SNP rs2201841 genotypes.

FIG. 5 shows levels of S100 proteins treatment responders and non-responders. For the upper part of the figure (panels A-C) the ASAS20 response definition was used while for the lower part (Panels D-F) ASAS40 was applied. Protein levels are shown for either S100A8 (left) or S100A9 (middle) individually or as the sum of S100A8+S100A9 (right).

FIG. 6 shows that, for AS patients, carriers of either of the ERAP1 risk alleles rs30187 or rs27434 typically showed higher levels of ERAP1 gene expression, while homozygous noncarriers mostly showed lower transcript levels.

### DETAILED DESCRIPTION OF THE DISCLOSURE

The term “assaying” is used to refer to the act of identifying, screening, probing, testing measuring or determining, which act may be performed by any conventional means. For example, a sample may be assayed for the presence of a particular genetic or protein marker by using an ELISA assay, a Northern blot, imaging, serotyping, cellular typing, gene sequencing, phenotyping, haplotyping, immunohistochemistry, western blot, mass spectrometry, etc. The term “detecting” (and the like) means the act of extracting particular information from a given source, which may be direct or indirect. In some embodiments of the predictive methods disclosed herein, the presence or absence of a given thing (e.g., allele, level of protein, etc.) is detected in a biological sample indirectly, e.g., by querying a database. The terms “assaying” and “determining” contemplate a transformation of matter, e.g., a transformation of a biological sample, e.g., a blood sample or other tissue sample, from one state to another by means of subjecting that sample to physical testing.

The term “obtaining” means to procure, e.g., to acquire possession of in any way, e.g., by physical intervention (e.g., biopsy, blood draw) or non-physical intervention (e.g., transmittal of information via a server), etc.

The phrase “assaying a biological sample . . . ” and the like is used to mean that a sample may be tested (either directly or indirectly) for either the presence or absence of a given factor (in the case of AS non-response alleles and AS response alleles) or for the level of a particular factor (in the case or AS response proteins). It will be understood that, in a situation where the presence of a substance denotes one probability and the absence of a substance denotes a different probability, then either the presence or the absence of such substance may be used to guide a therapeutic decision. For example, one may determine if a patient has an AS non-response allele by determining the actual existence of the AS non-response allele in the genome of a patient or by determining the absence of the AS non-response allele in the genome of a patient. In both such cases, one has determined whether the patient has the presence of the AS non-response allele.

The disclosed methods involve, inter alia, determining whether a particular individual has an AS non-response allele, an AS response allele and/or an AS response protein. In the case of AS non-response alleles or AS response alleles, this determination is undertaken by identifying whether the patient has the presence of an rs30187 non-response allele, an rs27434 non-response allele, an rs11209032 response allele or an rs2201841 response allele. Each of these determinations (i.e., presence or absence), on its own, provides the allelic status of the patient and thus each of these determinations equally provide an indication of whether a particular individual would or would not respond more favorably to IL-17 antagonism. In the case of AS response proteins, this determination is undertaken by measuring baseline levels of S100A8, S100A9 or S100A8+S100A9 protein in the subject.

It will be understood that patients heterozygous or homozygous for the AS non-response alleles disclosed herein (rs30187 non-response allele and rs27434 non-response allele) are less likely to respond favorably to IL-17 antagonism. Thus, to provide an indication of decreased responsiveness, a biological sample need only be assayed for one AS non-response allele, but clearly may be assayed for both AS non-response alleles. Similarly, it will be understood that patients heterozygous or homozygous for the AS response alleles disclosed herein (rs11209032 response allele and rs2201841 response allele) are more likely to respond favorably to IL-17 antagonism. Thus, to provide an indication of increased responsiveness, a biologically sample need only be assayed for one AS response allele, but clearly may be assayed for both AS response allele.

The term “about” in relation to a numerical value x means +/−10% unless the cotext dictates otherwise. The word “substantially” does not exclude “completely,” e.g., a composition which is “substantially free” from Y may be completely free from Y. Where necessary, the word “substantially” may be omitted from the definition of the disclosure.

“IL-17 antagonist” as used herein refers to a molecule capable of antagonizing (e.g., reducing, inhibiting, decreasing, delaying) IL-17 function, expression and/or signalling (e.g., by blocking the binding of IL-17 to the IL-17 receptor). Non-limiting examples of IL-17 antagonists include IL-17 binding molecules and IL-17 receptor binding molecules. In some embodiments of the disclosed methods, regimens, kits, processes, uses and compositions, an IL-17 antagonist is employed.

By “IL-17 binding molecule” is meant any molecule capable of binding to the human IL-17 antigen either alone or associated with other molecules. The binding reaction may be shown by standard methods (qualitative assays) including, for example, a binding assay, competition assay or a bioassay for determining the inhibition of IL-17 binding to its receptor or any kind of binding assays, with reference to a negative control test in which an antibody of unrelated specificity, but ideally of the same isotype, e.g., an anti-CD25 antibody, is used. Non-limiting examples of IL-17 binding molecules include small molecules, IL-17 receptor decoys, and antibodies as produced by B-cells or hybridomas and chimeric, CDR-grafted or human antibodies or any fragment thereof, e.g., F(ab′)₂ and Fab fragments, as well as single chain or single domain antibodies. Preferably the IL-17 binding molecule antagonizes (e.g., reduces, inhibits, decreases, delays) IL-17 function, expression and/or signalling. In some embodiments of the disclosed methods, regimens, kits, processes, uses and compositions, an IL-17 binding molecule is employed.

By “IL-17 receptor binding molecule” is meant any molecule capable of binding to the human IL-17 receptor either alone or associated with other molecules. The binding reaction may be shown by standard methods (qualitative assays) including, for example, a binding assay, competition assay or a bioassay for determining the inhibition of IL-17 receptor binding to IL-17 or any kind of binding assays, with reference to a negative control test in which an antibody of unrelated specificity, but ideally of the same isotype, e.g., an anti-CD25 antibody, is used. Non-limiting examples of IL-17 receptor binding molecules include small molecules, IL-17 decoys, and antibodies to the IL-17 receptor as produced by B-cells or hybridomas and chimeric, CDR-grafted or human antibodies or any fragment thereof, e.g., F(ab')₂ and Fab fragments, as well as single chain or single domain antibodies. Preferably the IL-17 receptor binding molecule antagonizes (e.g., reduces, inhibits, decreases, delays) IL-17 function, expression and/or signalling. In some embodiments of the disclosed methods, regimens, kits, processes, uses and compositions, an IL-17 receptor binding molecule is employed.

The term “antibody” as referred to herein includes whole antibodies and any antigen-binding portion or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments of the disclosed methods, regimens, kits, processes, uses and compositions, an antibody to IL-17 or the IL-17 receptor is employed, preferably an antibody to IL-17, e.g., secukinumab.

The term “antigen-binding portion” of an antibody as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen binding sites include the CDRs of secukinumab as set forth in SEQ ID NOs:1-6 and 11-13 (Table 3), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art. In some embodiments of the disclosed methods, regimens, kits, processes, uses and compositions, a single chain antibody or an antigen-binding portion of an antibody against IL-17 (e.g., secukinumab) or the IL-17 receptor is employed.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed methods, regimens, kits, processes, uses and compositions, the IL-17 antagonist is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof) binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by standard assays known in the art, such as by Biacore analysis.

As used herein, the terms “subject” and “patient” include any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least 10% of the measured parameter, by at least 50%, 80% or 90%, and in certain embodiments of the disclosed methods, uses, processes, kits and compositions, the IL-17 antibody used may inhibit greater than 95%, 98% or 99% of IL-17 functional activity.

“Inhibit IL-6” as used herein refers to the ability of an IL-17 antagonist (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) as disclosed herein typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods, regimens, kits, processes, uses and compositions, IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) and functional derivatives thereof have an IC₅₀ for inhibition of IL-6 production as defined above of about 20 nM or less, about 10 nM or less, about 5 nM or less, about 2 nM or less, or about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof) according to the present disclosure, e.g., of a specified sequence (e.g., a variable domain). A “functional derivative” includes a molecule having a qualitative biological activity in common with the disclosed IL-17 antagonista, e.g., IL-17 binding molecules. A functional derivative includes fragments and peptide analogs of an IL-17 antagonist as disclosed herein. Fragments comprise regions within the sequence of a polypeptide according to the present disclosure, e.g., of a specified sequence. Functional derivatives of the IL-17 antagonists disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 binding molecules disclosed herein (e.g., the V_{H} and/or V_{L} sequences of Table 3), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of a polypeptide according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” refer to both prophylactic or preventative treatment as well as curative or disease modifying treatment, including treatment of a patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected absent such treatment.

The phrase “respond to treatment” is used to mean that a patient, upon being delivered a particular treatment, e.g., an IL-17 binding molecule (e.g., secukinumab) shows a clinically meaningful benefit from said treatment. In the case of AS, such benefit may be measured by a variety of criteria, e.g., ASAS20, ASAS40, ASAS 5/6, BASDAI, etc. (see, e.g., Zoching et al (2007) Curr. Opin. Rheumatol. 19:346-50 and Example 1). All such criteria are acceptable measures of whether an AS patient is responding to a given treatment. The phrase “respond to treatment” is meant to be construed comparatively, rather than as an absolute response. For example, a patient having an AS non-response allele is predicted to have less benefit from treatment with an IL-17 antagonist than a patient who does not have an AS non-response allele. Similarly, a patient having an AS response allele is predicted to have more benefit from treatment with an IL-17 antagonist than a patient who does not have an AS response allele. These non-carriers of AS non-response alleles and carriers of AS response alleles respond more favorably to treatment with the IL-17 antagonist, and may simply be said to “respond to treatment” with an IL-17 antagonist.

As used herein, the phrase “ankylosing spondylitis” and its abbreviation “AS” refer to inflammatory arthridities characterized by chronic inflammation of joints, which can include the spine and the sacroilium in the pelvis, and which can cause eventual fusion of the spine. The modified New York criteria for AS or the ASAS axial SPA criteria (2009) may be used to diagnose a patient as having AS.

As used herein, “selecting” and “selected” in reference to a patient is used to mean that a particular patient is specifically chosen from a larger group of patients on the basis of (due to) the particular patient having a predetermined criteria, e.g., the patient does not have an AS non-response allele or the patient has an AS response allele. Similarly, “selectively treating a patient having AS” refers to providing treatment to an AS patient that is specifically chosen from a larger group of patients on the basis of (due to) the particular patient having a predetermined criteria, e.g., the patient does not have an AS non-response allele or the patient has an AS response allele. Similarly, “selectively administering” refers to administering a drug to an AS patient that is specifically chosen from a larger group of patients on the basis of (due to) the particular patient having a predetermined criteria, e.g., the patient does not have an AS non-response allele or the patient has an AS response allele. By selecting, selectively treating and selectively administering, it is meant that a patient is delivered a personalized therapy for AS based on the patient's biology, rather than being delivered a standard treatment regimen based solely on having the AS disease.

As used herein, “predicting” indicates that the methods described herein provide information to enable a health care provider to determine the likelihood that an individual having AS will respond to or will respond more favorably to treatment with an IL-17 binding molecule. It does not refer to the ability to predict response with 100% accuracy. Instead, the skilled artisan will understand that it refers to an increased probability.

As used herein, “likelihood” and “likely” is a measurement of how probable an event is to occur. It may be used interchangably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the IL-17 binding molecule or the patient may not be treated (or treatment discontinued, or treatment proceed with a lowered dose) with the IL-17 binding molecule.

The phrase “increased likelihood” refers to an increase in the probability that an event will occur. For example, some methods herein allow prediction of whether a patient will display an increased likelihood of responding to treatment with an IL-17 binding molecule or an increased likelihood of responding better to treatment with an IL-17 binding molecule in comparison to an AS patient who has an AS non-response allele or an AS patient who does not have an AS response allele.

The phrase “decreased likelihood” refers to a decrease in the probability that an event will occur. For example, the methods herein allow prediction of whether a patient will display a decreased likelihood of responding to treatment with an IL-17 binding molecule or a decreased likelihood of responding better to treatment with an IL-17 binding molecule in comparison to a AS patient who does not have an AS non-response allele or an AS patient who does not have an AS response allele.

As used herein “SNP” refers to “single nucleotide polymorphism”. A single nucleotide polymorphism is a DNA sequence variation occurring when a single nucleotide in the genome (or other shared sequence) differs between members of a biological species or paired chromosomes in an individual. Most SNPs have only two alleles, and one is usually more common in the population. A SNP may be present in an exon or an intron of a gene, an upstream or downstream untranslated region of a gene, or in a purely genomic location (i.e., non-transcribed). When a SNP occurs in the coding region of a gene, the SNP may be silent (i.e., a synonymous polymorphism) due to the redundancy of the genetic code, or the SNP may result in a change in the sequence of the encoded polypeptide (i.e., a non-synonymous polymorphism). In the instant disclosure, SNPs are identified by their Single Nucleotide Polymorphism Database (dbSNP) rs number, e.g., rs30187. The dbSNP is a free public archive for genetic variation within and across different species developed and hosted by the National Center for Biotechnology Information (NCBI) in collaboration with the National Human Genome Research Institute (NHGRI).

A polymorphic site, such as a SNP, is usually preceded by and followed by conserved sequences in the genome of the population of interest and thus the location of a polymorphic site can often be made in reference to a consensus nucleic acid sequence (e.g., of thirty to sixty nucleotides) that bracket the polymorphic site, which in the case of a SNP is commonly referred to as the “SNP context sequence”. Context sequences for the SNPs disclosed herein may be found in the NCBI SNP database available at: www.ncbi.nlm.nih.gov/snp. Alternatively, the location of the polymorphic site may be identified by its location in a reference sequence (e.g., GeneBank deposit) relative to the start of the gene, mRNA transcript, BAC clone or even relative to the initiation codon (ATG) for protein translation. The skilled artisan understands that the location of a particular polymorphic site may not occur at precisely the same position in a reference or context sequence in each individual in a population of interest due to the presence of one or more insertions or deletions in that individual as compared to the consensus or reference sequence. It is routine for the skilled artisan to design robust, specific and accurate assays for detecting the alternative alleles at a polymorphic site in any given individual, when the skilled artisan is provided with the identity of the alternative alleles at the polymorphic site to be detected and one or both of a reference sequence or context sequence in which the polymorphic site occurs. Thus, the skilled artisan will understand that specifying the location of any polymorphic site described herein by reference to a particular position in a reference or context sequence (or with respect to an initiation codon in such a sequence) is merely for convenience and that any specifically enumerated nucleotide position literally includes whatever nucleotide position the same polymorphic site is actually located at in the same locus in any individual being tested for the presence or absence of a genetic marker of the invention using any of the genotyping methods described herein or other genotyping methods well-known in the art.

In addition to SNPs, genetic polymorphisms include translocations, insertions, substitutions, deletions, etc., that occur in gene enhancers, exons, introns, promoters, 5′ UTR, 3′UTR, etc.

As shown in the Examples, we have determined that the ERAP1 (endoplasmatic reticulum aminopeptidase 1) rs30187 “T” allele and the ERAP1 rs27434 “A” alleles associate with reduced ASAS40 response during secukinumab treatment. “ERAP1” (also known as ARTS1) refers to the human ERAP1 gene, which encodes endoplasmic reticulum aminopeptidase, an enzyme involved in peptide antigen precursor trimming prior to presentation of antigens on MHC class 1 molecules. ERAP1 has also been demonstrated to promote shedding of the ectodomain of TNFR1, ILRII (decoy receptor) and IL-6R alpha, thereby influencing inflammatory signalling. The ERAP1 gene is located on chromosome 5, and certain ERAP1 SNPs have been shown to be associated with AS (Wellcome Trust et al (2007) Nat Genet. 39(11):1329-37).

As used herein, “rs30187” refers to a T/C SNP in the human ERAP1 gene located on chromosome 5, which is associated with both AS and multiple sclerosis (MS) (Wellcome Trust et al (2007) Nat Genet. 39(11):1329-37; M. Brown (2008) Rheumatology 47(2):132-7; Guerini et al. (2012) PLoS One 7(1)e29931). The rs30187 polymorphic site is located at chromosomal position 96,150,086 (NCBI genome build 36.3), position 30,519 of the human ERAP1 gene set forth as GeneBank Accession No. NG_{—}027839.1, position 1,688 of the human ERAP1 transcript variant 3 mRNA set forth as GeneBank Accession No. NM_{—}001198541, position 1,930 of the human ERAP1 transcript variant 2 mRNA set forth as GeneBank Accession No. NM_{—}001040458; codon encoding amino acid 528 of the human ERAP1 protein set forth as GeneBank Accession No. NP_{—}057526.3). The rs30187 C allele encodes a Lys528Arg variant of ERAP1 that has decreased catalytic properties, and is associated with a decreased incidence of AS. (Kochan et al. (2011) Proc Natl Acad Sci USA. 108(19):7745-50). The rs30187 SNP is one of several SNPs in the ERAP1 gene that have been shown to be associated with AS. (See Wellcome Trust et al (2007) Nat Genet. 39(11):1329-37; M. Brown (2008) Rheumatology 47(2):132-7). Other ERAP1 SNPs associated with AS include rs27044, rs27434, rs17482078, rs10050860, and rs2287987. Each “T” disease allele of rs30187 increases the odds of having AS by about 1.4× (Table 1a).

As used herein, the term “rs27434” refers to an A/G SNP in the human ERAP1 gene. (Lin et al. (2011) J Rheumatol. 38(2):317-21). The rs27434 polymorphic site is located at position 25,337 of the human ERAP1 gene set forth as GeneBank Accession No. NG_{—}027839.1 (position 1,415 of the human ERAP1 mRNA set forth as GeneBank Accession No. NM_{—}016442.3; codon encoding amino acid 356 of the human ERAP1 protein set forth as GeneBank Accession No. NP_{—}057526.3). The rs27434 SNP is a synonymous polymorphism occurring in the codon for Ala356 of the ERAP1 protein. (Harvey et al. (2009) Hum. Mol. Genet. 18 (21): 4204-4212). Each “A” disease allele of rs27434 increases the odds of having AS by about 1.19× (Table 1a).

The phrase “AS non-response allele” as used herein refers to the T allele (A allele, in the case of the noncoding strand) at the rs30187 polymorphic site (“rs30187 non-response allele”), and the A allele (T allele, in the case of the noncoding strand) at the rs27434 polymorphic site (“rs27434 non-response allele. These alleles are shown in Table 1a. In some embodiments of the disclosed methods, uses, and kits, the patient has at least one AS non-response allele.

TABLE 1a
AS non-response alleles.

| | | Allele | Frequency of | Poly- | Odds |
|---|---|---|---|---|---|
| | | (disease/non- | disease allele | morphism | ratio |
| Rs # | Gene | disease | in population | position | for AS |
| 27434 | ERAP1 | A/G | 0.23 | synonymous | 1.19 |
| 30187 | ERAP1 | T/C | 0.34 | non- | 1.40 |
| | | | | synonymous | |

TABLE 1b
AS response alleles.

| | | Alleles | Frequency of | Poly- | Odds |
|---|---|---|---|---|---|
| | | (disease/non- | disease allele | morphism | ratio |
| Rs # | Gene | disease | in population | position | for AS |
| 11209032 | IL23R | A/G | 0.32 | downstream | 1.30 |
| 2201841 | IL23R | C/T | 0.28 | intronic | 2.12* |

*= odds ratio calculated based on CC vs. CT + TT, not based on C allele.

For Table 1a and 1b, the odds ratios (column 6) may be found as follows: for rs27434: TASC et al (2010) Nat Genet. 42(2):123-2; for rs30187: Wellcome Trust et al (2007) Nat Genet. 39(11):1329-37; for rs11209032: Wellcome Trust et al (2007) Nat Genet. 39(11):1329-37; for rs2201841: Safrany et al (2009) Scand J Immunology 70(1):68-74.

As shown in the Examples, following secukinumab treatment, patients having at least one IL23R (Interleukin-23 receptor) rs11209032 “G” allele display improved Bath Ankylosing Spondylitis Disease Activity Index (BASDAI) scores over time relative to patients having only the rs11209032 “A” allele, and patients having at least one rs2201841 “T” allele display improved BASDAI scores over time relative to patients having only the rs2201841 “C” allele. “IL23R” refers to the human interleukin 23 receptor gene, which encodes the receptor for the IL-23 cytokine, a cytokine produced by dendritic cells and macrophages, and which promotes upregulation of the matrix metalloprotease MMP9, increases angiogenesis and reduces CD8+ T-cell infiltration. In conjunction with IL-6 and TGF-β1, IL-23 stimulates naive CD4+ T cells to differentiate into a novel subset of cells called Th17 cells, which are distinct from the classical Th1 and Th2 cells. Th17 cells produce IL-17, a proinflammatory cytokine that enhances T cell priming and stimulates the production of proinflammatory molecules such as IL-1, IL-6, TNF-alpha, NOS-2, and chemokines resulting in inflammation. The IL23R gene is located on chromosome 1, and certain IL23R SNPs have been shown to be associated with AS and RA (Wellcome Trust et al (2007) Nat Genet. 39(11):1329-37; Farago et al. (2008) Ann Rheum Dis. 67(2):248-50).

As used herein, “rs11209032” refers to an A/G SNP downstream of the human IL23R gene that is associated with AS. (Wellcome Trust et al (2007) Nat Genet. 39(11):1329-37; M. Brown (2008) Rheumatology 47(2):132-7; Lee et al. (2012) Inflamm. Res. 61(2):143-9). The rs11209032 polymorphic site is located at position 67740092 of GRCh37.p5 (Genome Reference Consortium Human genome build 37). The rs11209032 SNP is one of several SNPs near or in the IL23R gene that have been shown to be associated with AS. (See Wellcome Trust et al (2007) Nat Genet. 39(11):1329-37; M. Brown (2008) Rheumatology 47(2):132-7). Other IL23R SNPs associated with AS include rs11209026, rs1004819, rs10489629, rs11465804, rs1343151, rs10889677. Each “A” disease allele of rs11209032 increases the odds of having AS by about 1.3× (Table 1b).

As used herein, “rs2201841” refers to a C/T SNP located within an intron of the human IL23R gene that is associated with AS. (Lee et al. (2012) Inflamm. Res. 61(2):143-9). The rs11209032 polymorphic site is located at position 67694202 of GRCh37.p5, which is position 67,034 of the human IL23R gene set forth as GeneBank Accession No. NG_{—}011498.1. The “CC” genotype of rs2201841 increases the odds of having AS by about 2.12 (Table 1b).

As used herein the phrase “rs11209032 (A;G) genotype” refers to a heterozygous genotype in which the patient has one A allele and one G allele at the rs11209032 polymorphic site, the phrase “rs11209032 (A;A) genotype” refers to a homozygous genotype in which the patient has two A alleles at the rs11209032 polymorphic site, and the phrase “rs11209032 (G;G) genotype” refers to a homozygous genotype in which the patient has two G alleles at the rs11209032 polymorphic site. As used herein the phrase “rs2201841 (C;T) genotype” refers to a heterozygous genotype in which the patient has one C allele and one T allele at the rs2201841 polymorphic site, the phrase “rs2201841 (C;C) genotype” refers to a homozygous genotype in which the patient has two C alleles at the rs2201841 polymorphic site, and the phrase “rs2201841 (T;T) genotype” refers to a homozygous genotype in which the patient has two T alleles at the rs2201841 polymorphic site. In some embodiments of the disclosed methods, uses, and kits, the patient is of the rs11209032 (A;G) genotype, the rs11209032 (A;A) genotype, or the rs11209032 (G;G) genotype. In some embodiments of the disclosed methods, uses, and kits, the patient has the rs2201841 (C;T) genotype, the rs2201841 (C;C) genotype, or the rs2201841 (T;T) genotype.

The phrase “AS response allele” as used herein refers to the G allele (C allele, in the case of the noncoding strand) at the rs11209032 polymorphic site (“rs11209032 response allele”), and the T allele (A allele, in the case of the coding strand) at the rs2201841 polymorphic site (“rs2201841 response allele”). These alleles are shown in Table 1b. In some embodiments of the disclosed subject matter, the patient has at least one AS response allele.

As recognized by the skilled artisan, nucleic acid samples containing a particular SNP may be complementary double stranded molecules and thus reference to a particular site on the sense strand refers as well to the corresponding site on the complementary antisense strand. Similarly, reference to a particular genotype obtained for a SNP on both copies of one strand of a chromosome is equivalent to the complementary genotype obtained for the same SNP on both copies of the other strand. Thus, for example, a C/C genotype for the rs30187 polymorphic site on the coding strand for the ERAP1 gene is equivalent to a G/G genotype for that polymorphic site on the noncoding strand.

As used herein, the phrase “AS risk marker” refers to the polymorphic sites and alleles shown in Table 2, which may be used to further stratify patients having an increased (or decreased) likelihood of responding to treatment with an IL-17 antagonist. It will be understood that the AS risk markers in Table 2 can be used alone or in combination with the AS non-response alleles and AS response alleles of Table 1 to predict response of an AS patient to an IL-17 binding molecule, e.g., secukinumab. In some embodiments, a biological sample from a patient is assayed for the presence of an AS non-response allele and/or an AS response allele and, optionally, an AS risk marker. The AS risk marker alleles associated with AS disease in Table 2 are shown in bolded larger font in column 3.

TABLE 2
AS Risk Markers.

| | | Alleles | |
|---|---|---|---|
| Variant | Gene | (minor/major) | Polymorphism position |
| rs11209026 | IL23R | A/G | non-synonymous |
| rs10865331 | — | A/G | genomic |
| rs2310173 | IL1R2 | A/C | downstream |
| rs4333130 | ANTXR2 | C/T | intronic |
| rs2242944 | — | A/G | genomic |
| rs1974226 | IL17A | T/C | 3′ UTR |
| rs7747909 | IL17A | A/G | 3′ UTR |
| HLA-DRB1*04 | HLA-DRB1 | | |
| HLA-B*27 | HLA-B | | |

As used herein “S100A8” refers to the human protein (also known as migration inhibitory factor-related protein 8 (MRP-8) or calgranulin-A) encoded by the human S100A8 gene. The protein encoded by this gene is a member of the S100 family of proteins containing 2 EF-hand calcium-binding motifs. S100 proteins are localized in the cytoplasm and/or nucleus of a wide range of cells, and involved in the regulation of a number of cellular processes such as cell cycle progression and differentiation. S100 genes include at least 13 members which are located as a cluster on chromosome 1q21. This protein may function in the inhibition of casein kinase and as a cytokine. As used herein, “S100A9” refers to the human protein (also known as migration inhibitory factor-related protein 14 (MRP-14) or calgranulin-B) encoded by the human S100A9 gene. S100A8 complexes with S100A9 (synonyma: S100A8/S100A9, S100A8+S100A9, Calgranulin A/B, Calprotectin) to, inter alia, regulate myeloid cell function by binding to Toll-like receptor and the receptor for advanced glycation end products (Boyd et al. (2008) Circ. Res. 102(10)1239-46), regulate vascular inflammation and promote leukocyte recruitment (Croce et al. (2009) Circulation 120(5):427-36), and control neutrophil and macrophage accumulation, macrophage cytokine production, and SMC proliferation. Monomeric forms of S100A8 and S100A9, as well as calgranulin, are marker proteins for certain inflammatory diseases in humans, especially rheumatoid arthritis (Baillet et al. (2010) Rheumatology 49:671-82; Chang et al. (2009) J. Rheumatol. 36:872-880; de Seny et al. (2008) Clin. Chem. 54(6):1066-75; Tilleman et al. (2005) Proteomics 5(8):2247-57). It has recently been reported that calprotectin levels, while frequently elevated in feces of AS patients, are normal in the serum of most AS patients (Klingberg et al., Scand. J. Gastro., Early Online 1-10 (Jan. 10, 2012). As used herein, the term “S100A9+S100A8” refers to the sume of S100A8 levels and S100A9 levels.

As shown in the Examples, we have determined that elevated baseline serum levels of S100A8, S100A9 and S100A8+S100A associate with increased ASAS20 and 40 response during secukinumab treatment. The phrase “AS response protein” includes S100A8, S100A9, and S100A8+S100A9. When assessing the level of S100A8+S100A9, the level of S100A8 individually and the level of S100A9 individually may be summed. Thus, for example, a comparison of a test level of S100A8+S100A9 to a control level of S100A8+S100A9 could be achieved by comparing the sum of the level of S100A8 and the level of S100A9 in a test sample to the sum of the level of S100A8 and the level of S100A9 in a control sample. As an alternative, one may assess the level of S100A8+S100A9 in a sample by directly measuring the level of the non-reduced S100A8+S100A9 complex, e.g., using an ELISA assay. In some embodiments, the subject has an increased level of S100A8, S100A9, and/or S100A8+S100A9 in comparison to a control.

The levels of S100A8, S100A9, and S100A8+S100A9 in test samples and control samples can be determined by analyzing the level of S100A8 and/or S100A9 protein in such samples. A “test level” is derived (directly or indirectly) from a biological sample from an AS patient of interest. A “control level” is derived (directly or indirectly) from a control biological sample or a predetermined reference standard (e.g., a reference concentration of the analyte in IL-17 antagonist non-responder patients or in the general population). A control biological sample is derived (directly or indirectly) from a biological sample obtained from an AS patient known not to respond to treatment with an IL-17 antagonist (“IL-17 antagonist non-responder”). Useful, nonlimiting, controls include, e.g., reference concentrations of the AS response proteins in non-responders, average or mean concentration of the AS response protein in matched samples from a population of non-responders, a concentration of the AS response protein below which, e.g., 95% of nonresponders fall, or a particular level of protein in a spiked surrogate-matrix, which reflects the levels in non-responders. A surrogate matrix is defined as a matrix that does not contain an analyte of interest, but otherwise reflects the attributes of the original matrix as close as possible (e.g., plasma). The analyte (e.g., S100A8+S100A9) is then spiked at a defined concentration into this matrix. In some embodiments of the disclosed methods, uses, and kits, the control level is derived from a predetermined reference standard or a control biological sample from an IL-17 non-responder.

The comparison of test and control levels of AS response proteins allows determination of whether the test level of an AS response protein (or combination thereof) is greater than or lower than a control level of a corresponding AS response protein (or combination thereof). As used herein, “greater than” means a larger value. As used herein, “lower than” means a smaller value. In some embodiments, the test level is greater than the control level. In some embodiments, the test level is at least about 20%, 25%, 30%, 35%, 40%, 45%, 50%, 55%, 60%, 65%, 70%, 75%, 80%, 85%, 90%, 95%, 100% greater than the control level.

In some embodiments, a biological sample from a patient is assayed for the level of S100A8, S100A9, or S100A8 and S100A9 as well as the presence of an AS non-response allele and/or an AS response allele (and optionally an AS risk marker).

As used herein, “genomic sequence” refers to a DNA sequence present in a genome, and includes a region within an allele, an allele itself, or a larger DNA sequence of a chromosome containing an allele of interest. Products of the AS non-response alleles, AS risk markers, AS risk proteins and AS response alleles include nucleic acid products and polypeptide products. “Polypeptide product” refers to a polypeptide encoded by an AS non-response allele or AS response allele and fragments thereof “Nucleic acid product” refers to any DNA (e.g., genomic, cDNA) or RNA (e.g., pre-mRNA, mRNA, miRNA) products of an AS non-response allele or AS response allele and fragments thereof. In the context of AS risk proteins, a “polypeptide product” refers to a S100A8, S100A9 or S100A8+S100A9 protein or fragment thereof.

An “equivalent genetic marker” refers to a genetic marker that is correlated to an allele of interest, e.g., it displays linkage disequilibrium (LD) or is in genetic linkage with the allele of interest. Equivalent genetic markers may be used to determine if a patient has an AS non-response allele and/or an AS response allele, rather than directly interrogating a biological sample from the patient for the AS non-response allele and/or an AS response allele per se. Information on the extensive LD within and surrounding ERAP1 may be found, e.g., in Harvey et al. (2009) Hum. Mol. Genet. 18(21):4204-4012.

The term “probe” refers to any composition of matter that is useful for specifically detecting another substance, e.g., a substance related to an AS non-response allele or an AS response protein. A probe can be an oligonucleotide (including a conjugated oligonucleotide) that specifically hybridizes to a genomic sequence of an AS non-response allele or a nucleic acid product of an AS non-response allele (e.g., mRNA). A conjugated oligonucleotide refers to an oligonucleotide covalently bound to chromophore or molecules containing a ligand (e.g., an antigen), which is highly specific to a receptor molecule (e.g., an antibody specific to the antigen). The probe can also be a PCR primer, e.g., together with another primer, for amplifying a particular region within an AS non-response allele or an AS response allele. Further, the probe can be an antibody that specifically binds to an AS non-response allele, a polypeptide product of an AS non-response allele, an AS response allele or an AS response protein. Further, the probe can be any composition of matter capable of detecting (e.g., binding or hybridizing) an equivalent genetic marker of an AS non-response allele or an AS response allele. In preferred embodiments, the probe specifically hybridizes to a nucleic acid sequence or specifically binds to a polypeptide sequence.

The phrase “specifically hybridizes” is used to refer to hybridization under stringent hybridization conditions. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5 M sodium phosphate, 7% SDS at 65° C., followed by at least one wash at 0.2×SSC, 1% SDS at 65° C.

The phrase “a region of a nucleic acid” is used to indicate a smaller sequence within a larger sequence of nucleic acids. For example, a gene is a region of a chromosome, an exon is a region of a gene, etc.

The term “specifically binds” in the context of polypeptides is used to mean that a probe binds a given polypeptide target (e.g., a polypeptide product of an AS non-response allele or an AS response protein) rather than randomly binding undesirable polypeptides. However, “specifically binds” does not exclude some cross reactivity with undesirable polypeptides, as long as that cross reactivity does not interfere with the capability of the probe to provide a a useful measure of the presence or absence of the given polypeptide target.

The term “capable” is used to mean that ability to achieve a given result, e.g., a probe that is capable of detecting the presence of a particular substance means that the probe may be used to detect the particular substance.

An “oligonucleotide” refers to a short sequence of nucleotides, e.g., 2-100 bases.

The term “biological sample” as used herein refers to a sample from a patient, which may be used for the purpose of identification, diagnosis, prediction, or monitoring. Preferred test samples include synovial fluid, blood, blood-derived product (such as buffy coat, serum, and plasma), lymph, urine, tear, saliva, cerebrospinal fluid, buccal swabs, feces, hair bulb cells, synovial fluid, synovial cells, sputum, or tissue samples. In addition, one of skill in the art would realize that some test samples would be more readily analyzed following a fractionation or purification procedure, for example, isolation of DNA from whole blood.

The phrases “has been previously treated for AS” and “had a previous AS treatment” and the like are used to mean a patient that has previously undergone AS threapy, e.g, using an AS agent, e.g., the patient is a failure, an inadequate responder, or intolerant to a previous AS therapy, anti-AS agent or treatment regimen. Such patients include those previously treated with, e.g., an NSAID, a TNF alpha antagonist, sulfasalazine, methotrexate, a corticosteroid or combinations thereof. The phrase “has not been previously treated for AS” is used to mean a patient that has not previously undergone AS treatment, i.e., the patient is “naïve.” As used herein, a patient that has not been previously treated for AS with a TNF alpha antagonist is deemed “TNF alpha antagonist naive”. As used herein, the phrase “AS agent” refers to pharmaceuticals commonly prescribed for AS patients, e.g., NSAIDs (e.g., indomethacin, naproxen, sulindac, diclofenac, aspirin, flurbiprofen, oxaprozin, salsalate, difunisal, piroxicam, etodolac, meclofenamate, ibuprophen, fenoprofen, ketoprofen, nabumetone, tolmetin, cholin magnesium salicylate, COX-2 inhibitors [e.g., celecoxib]), TNF alpha antagonists (etanercept, adalimumab, infliximab, golimumab), DMARDS (e.g., sulfasalazine, methotrexate), and corticosteroids.

The term “failure” to a previous AS therapy refers to: (1) a patient who has no meaningful clinical benefit (primary lack of efficacy) (also termed a “non-responder”); (2) a patient who has a measurable and meaningful response, but for whom response could be better, e.g., low AS disease activity or AS remission was not achieved (also termed “inadequate response” or “incomplete response”); (3) a patient who, after an initial good response, worsens (secondary loss of efficacy); and (4) a patient who has a good response but discontinues because of a side effect (also termed “intolerance”). Patients who show TNF alpha antagonist incomplete response (TNF-IR) or intolerance to TNF alpha antagonists are considered TNF alpha antagonist failures. Patients who show sulfasalazine inadequate response (SFS-IR) or intolerance to sulfasalazine are considered sulfasalazine failures. Patients who show DMARD inadequate response (DMARD-IR) or intolerance to DMARDs are considered DMARD failures. Patients who show NSAID inadequate response (NSAID-IR) or intolerance to NSAIDs are considered NSAID failures. In some embodiments of the disclosed methods, the patient is a TNF failure (e.g., a TNF-IR patient) or is TNF naive.

### IL-17 Antagonists

The various disclosed pharmaceutical compositions, regimens, processes, uses, methods and kits utilize an IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof).

In one embodiment, the IL-17 antagonist, e.g., IL-17 binding molecule (e.g., IL-17 antibody or antigen binding fragment thereof, e.g., secukinumab) comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antagonist, e.g., IL-17 binding molecule (e.g., IL-17 antibody or antigen binding fragment thereof, e.g., secukinumab) comprises at least one immunoglobulin light chain variable domain (V_{L′}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antagonist, e.g., IL-17 binding molecule (e.g., IL-17 antibody or antigen binding fragment thereof, e.g., secukinumab) comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antagonist, e.g., IL-17 binding molecule (e.g., IL-17 antibody or antigen binding fragment thereof, e.g., secukinumab) comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antagonist, e.g., IL-17 binding molecule (e.g., IL-17 antibody or antigen binding fragment thereof, e.g., secukinumab) comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO:8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 3, below.

TABLE 3
Amino acid sequences of the hypervariable regions
of the secukinumab monoclonal antibodies.
Light-Chain

| | | |
|---|---|---|
| CDR1′ | Kabat | R-A-S-Q-S-V-S-S-S-Y-L-A |
| | | (SEQ ID NO: 4) |
| | Chothia | R-A-S-Q-S-V-S-S-S-Y-L-A |
| | | (SEQ ID NO: 4) |
| CDR2′ | Kabat | G-A-S-S-R-A-T (SEQ ID NO: 5) |
| | Chothia | G-A-S-S-R-A-T (SEQ ID NO: 5) |
| CDR2′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: 11) |
| CDR2 | Kabat | A-I-N-Q-D-G-S-E-K-Y-Y-V-G-S-V-K-G |
| | | (SEQ ID NO: 2) |
| CDR2-x | Chothia | A-I-N-Q-D-G-S-E-K-Y-Y |
| | | (SEQ ID NO: 12) |
| CDR3 | Kabat | D-Y-Y-D-I-L-T-D-Y-Y-I-H-Y-W-Y-F-D-L |
| | | (SEQ ID NO: 3) |
| CDR3-x | Chothia | C-V-R-D-Y-Y-D-I-L-T-D-Y-Y-I-H-Y-W- |
| | | Y-F-D-L-W-G (SEQ ID NO: 13) |

In preferred embodiments, the constant region domains preferably also comprise suitable human constant region domains, for instance as described in “Sequences of Proteins of Immunological Interest”, Kabat E. A. et al, US Department of Health and Human Services, Public Health Service, National Institute of Health. DNA encoding the VL of secukinumab is set forth in SEQ ID NO:9, and DNA encoding the VH of secukinumab is set forth in SEQ ID NO:7.

In some embodiments, the IL-17 antagonist, e.g., IL-17 binding molecule (e.g., IL-17 antibody or antigen binding fragment thereof, e.g., secukinumab) comprises the three CDRs of SEQ ID NO:10. In other embodiments, the IL-17 antagonist comprises the three CDRs of SEQ ID NO:8. In other embodiments, the IL-17 antagonist comprises the three CDRs of SEQ ID NO:10 and the three CDRs of SEQ ID NO:8. CDRs of SEQ ID NO:8 and SEQ ID NO:10, according to both the Chothia and Kabat definition, may be found in Table 3.

In some embodiments, the IL-17 antagonist, e.g., IL-17 binding molecule (e.g., IL-17 antibody or antigen binding fragment thereof, e.g., secukinumab) comprises the light chain of SEQ ID NO:15. In other embodiments, the IL-17 antagonist comprises the heavy chain of SEQ ID NO:17. In other embodiments, the IL-17 antagonist comprises the light chain of SEQ ID NO:15 and the heavy domain of SEQ ID NO:17. In some embodiments, the IL-17 antagonist comprises the three CDRs of SEQ ID NO:15. In other embodiments, the IL-17 antagonist comprises the three CDRs of SEQ ID NO:17. In other embodiments, the IL-17 antagonist comprises the three CDRs of SEQ ID NO:15 and the three CDRs of SEQ ID NO:17. CDRs of SEQ ID NO:15 and SEQ ID NO:17, according to both the Chothia and Kabat definition, may be found in Table 3. The DNA encoding the light chain of secukinumab is set forth as SEQ ID NO:14. The DNA encoding the heavy chain of secukinumab is set forth as SEQ ID NO:16.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antagonist, e.g., IL-17 binding molecule (e.g., IL-17 antibody or antigen binding fragment thereof, e.g., secukinumab) is selected from a human anti IL-17 antibody which comprises at least: a) an immunoglobulin heavy chain or fragment thereof which comprises a variable domain comprising in sequence the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising in sequence the hypervariable regions CDR1′, CDR2′, and CDR3′ and the constant part or fragment thereof of a human light chain, said CDR1′ having the amino acid sequence SEQ ID NO: 4, said CDR2′ having the amino acid sequence SEQ ID NO:5, and said CDR3′ having the amino acid sequence SEQ ID NO:6.

In one embodiment, the IL-17 antagonist, e.g., IL-17 binding molecule (e.g., IL-17 antibody or antigen binding fragment thereof, e.g., secukinumab) is selected from a single chain binding molecule which comprises an antigen binding site comprising: a) a first domain comprising in sequence the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antagonist, e.g., IL-17 binding molecule (e.g., IL-17 antibody or antigen binding fragment thereof) for use in the disclosed methods may comprise a derivative of the IL-17 binding molecules set forth herein by sequence (e.g., a pegylated version of secukinumab). Alternatively, the V_{H} or V_{L} domain of an IL-17 antagonist, e.g., IL-17 binding molecule (e.g., IL-17 antibody or antigen binding fragment thereof) for use in the disclosed methods may have V_{H} or V_{L} domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:17 and/or a light chain that is substantially identical to that set forth as SEQ ID NO:15. A human IL-17 antibody disclosed herein may comprise a heavy chain that comprises SEQ ID NO:17 and a light chain that comprises SEQ ID NO:15. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain. Alternatively, an IL-17 antagonist, e.g., IL-17 binding molecule (e.g., IL-17 antibody or antigen binding fragment thereof) for use in the disclosed methods may be an amino acid sequence variant of the reference IL-17 binding molecules set forth herein. In all such cases of derivative and variants, the IL-17 antagonist is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts.

The inhibition of the binding of IL-17 to its receptor may be conveniently tested in various assays including such assays as described in WO 2006/013107. By the term “to the same extent” is meant that the reference and the derivative molecules exhibit, on a statistical basis, essentially identical IL-17 inhibitory activity in one of the assays referred to herein (see Example 1 of WO 2006/013107). For example, the IL-17 binding molecules disclosed herein typically have IC₅₀s for the inhibition of human IL-17 on IL-6 production induced by human IL-17 in human dermal fibroblasts which are below about 10 nM, more preferably about 9, 8, 7, 6, 5, 4, 3, 2, or about 1 nM of that of, preferably substantially the same as, the IC₅₀ of the corresponding reference molecule when assayed as described in Example 1 of WO 2006/013107. Alternatively, the assay used may be an assay of competitive inhibition of binding of IL-17 by soluble IL-17 receptors (e.g. the human IL-17 R/Fc constructs of Example 1 of WO 2006/013107) and the IL-17 antagonists of the disclosure.

The disclosure also includes IL-17 antagonists, e.g., IL-17 binding molecules (e.g., IL-17 antibody or antigen binding fragment thereof, e.g., secukinumab) in which one or more of the amino acid residues of CDR1, CDR2, CDR3, CDR1-x, CDR2-x, CDR3-x, CDR1′, CDR2′ or CDR3′ or the frameworks, typically only a few (e.g., 1-4), are changed; for instance by mutation, e.g., site directed mutagenesis of the corresponding DNA sequences. The disclosure includes the DNA sequences coding for such changed IL-17 antagonists. In particular the disclosure includes IL-17 binding molecules in which one or more residues of CDR1′ or CDR2′ have been changed from the residues shown in SEQ ID NO:4 (for CDR1′) and SEQ ID NO:5 (for CDR2′).

The disclosure also includes IL-17 antagonists, e.g., IL-17 binding molecules (e.g., IL-17 antibody or antigen binding fragment thereof, e.g., secukinumab) that have binding specificity for human IL-17, in particular IL-17 antibodies capable of inhibiting the binding of IL-17 to its receptor and IL-17 antibodies capable of inhibiting the activity of 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50% (said inhibitory activity being measured on IL-6 production induced by hu-IL-17 in human dermal fibroblasts).

In some embodiments, the IL-17 antagonist, e.g., IL-17 antibody, e.g., secukinumab, binds to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define these epitopes is based on residue one being the first amino acid of the mature protein (ie., IL-17A lacking the 23 amino acid N-terminal signal peptide and beginning with Glycine). The sequence for immature IL-17A is set forth in the Swiss-Prot entry Q16552. In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (s.c.) administered IL-17 antibody has a range of about 60-about 80%, e.g., about 76%. In some embodiments, the IL-17 antagonist, e.g., an IL-17 binding molecule (e.g., an IL-17 antibody, such as secukinumab) or an IL-17 receptor binding molecule (e.g., an IL-17 receptor antibody) has an elimination half-life of about 4 weeks (e.g., about 23 to about 35 days, about 23 to about 30 days, e.g., about 30 days). In some embodiments, the IL-17 antagonist, e.g., an IL-17 binding molecule (e.g., an IL-17 antibody, such as secukinumab) or an IL-17 receptor binding molecule (e.g., an IL-17 receptor antibody) has a Tₘₐₓ of about 7-8 dAS.

Particularly preferred IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) for use in the disclosed methods, uses, kits, etc. are human antibodies, especially secukinumab as described in Examples 1 and 2 of WO 2006/013107. Secukinumab is a recombinant high-affinity, fully human monoclonal anti-human interleukin-17A (IL-17A, IL-17) antibody of the IgG1/kappa isotype that is currently in clinical trials for the treatment of immune-mediated inflammatory conditions. Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, secukinumab has a very long half life, i.e., about 4 weeks, which allows for prolonged periods between administration, an exceptional property when treating chronic life-long disorders, such as rheumatoid arthritis (RA).

Other preferred IL-17 antagonists for use in the disclosed methods, kits and uses are those set forth in U.S. Pat. Nos. 8,057,794; 8,003,099; 8,110,191; and 7,838,638 and US Published Patent Application Nos: 20120034656 and 20110027290.

### Techniques for Assaying, Diagnostic Methods and Methods of Producing a Transmittable Form of Information

The disclosed methods are useful for the treatment, prevention, or amelioration AS, as well as predicting the likelihood of an AS patient's response to treatment with an IL-17 antagonist, e.g., secukinumab. These methods employ, inter alia, detecting whether a patient has the presence (or absence) of an AS non-response allele, an AS response allele, determining the level of an AS response protein in a sample from the patient, and/or determining the level of ERAP1 expression, level of ERAP1 protein or level of ERAP1 activity.

When the step of detecting is performed by assaying a biological sample from the patient, assaying may be performed by any conventional means, which will be selected depending on whether the particular marker falls within an exon, an intron, a non-coding portion of mRNA or a non-coding genomic sequence.

Numerous sources may be used to identify the presence or absence of alleles or proteins, the level of expression of genes or proteins, and the activity of a protein, e.g., blood, synovial fluid, buffy coat, serum, plasma, lymph, feces, urine, tear, saliva, cerebrospinal fluid, buccal swabs, sputum, or tissue. Various sources within a biological sample may be used in the disclosed methods, e.g., one may assay genomic DNA obtained from a biological sample to detect alleles or one may assay nucleic acid products (e.g., DNA, pre-mRNA, mRNA, micro RNAs, etc.) and polypeptide products (e.g., expressed proteins) of alleles.

We have determined that the ERAP1 rs30187 “T” allele and the ERAP1 rs27434 “A” allele associate with reduced ASAS40 response during secukinumab treatment. We have also determined that following secukinumab treatment, patients having at least one IL23R rs 11209032 “G” allele display improved BASDAI scores over time relative to patients having only the rs11209032 “A” allele, and patients having at least one rs2201841 “T” allele display improved BASDAI scores over time relative to patients having only the rs2201841 “C” allele. We thus contemplate that testing subjects for the presence of one or more of these ERAP1 or IL23R alleles will be useful in a variety of pharmacogenetic products and methods that involve identifying individuals more likely to respond to IL-17 antagonsim therapy and in helping physicians decide whether to prescribe IL-17 antagonists (e.g., secukinumab) to a patient having AS. Because rs30187 falls within the ERAP1 coding region and causes an amino acid change in the ERAP1 protein, the transcribed ERAP1 mRNA and translated ERAP1 protein differ between subjects having the rs30187 non-response allele and the rs30187 response allele. It may also be possible to determine the presence of the rs30187 non-response allele by measurement of the proteolytic activity of the ERAP1 protein, as the rs30187 variant has been shown to affect the ERAP1 proteolytic activity. Polymorphic site rs27434 also falls within the ERAP1 coding region, and so the transcribed mRNA differs between subjects having the rs27434 non-response allele and subjects having the rs27434 response allele. However, the rs27434 SNP is a synonymous polymorphism, and one cannot detect a patient's rs27434 allelic status by analyzing the sequence of the ERAP1 protein.

The rs2201841 SNP is found in an IL23R intron, such that a patient's allelic status may be determined by interrogating, e.g., pre-mRNA or genomic DNA. The rs11209032 SNP is found downstream of the IL23R gene, such that a patient's allelic status may be determined by interrogating, e.g., genomic DNA. Accordingly, a skilled artisan will understand that one may identify whether a subject has an AS non-response allele or AS response allele (or an AS risk marker) by assaying a genomic sequence of the AS non-response allele, a nucleic acid product of a AS non-response allele (e.g., pre-mRNA, mature mRNA, microRNA, cDNA made from mRNA, etc.), a polypeptide product of an AS non-response allele (in the case of rs30187), or an equivalent genetic marker of the AS non-response allele or an AS response allele. In preferred embodiments, a genomic sequence or a nucleic acid product of an AS non-response allele or an AS response allele is analyzed to determine whether a subject has an AS non-response allele or an AS response allele.

Our work shows that AS carriers of either of the ERAP1 rs30187 “T” allele or rs27434 “A” allele have decreased response to IL-17 antagonism, and that AS carriers of these alleles typically showed higher levels of ERAP1 gene expression. This suggests to us that decreased ERAP1 expression may be predictive of an improved response to IL-17 antagonism for AS patients. Furthermore, the rs30187 protective allele “C” encodes an ERAP1 protein variant (K528R) with reduced catalytic activity relative to the ERAP1 protein encoded by the rs30187 risk allele “T” (Kochan et al. (2011) Proc Natl Acad Sci USA. 108(19):7745-50). Thus, our work also suggests that decreased levels of ERAP1 protein or activity may be predictive of improved response to IL-17 antagonism for AS patients. Levels of ERAP1 expression, ERAP1 protein and/or ERAP1 activity may be directly measured by various techniques disclosed herein. In addition, any ERAP1 polymorphism (e.g., translocations, insertions, substitutions, deletions, SNP, etc., that occur in ERAP1 enhancers, exons, introns, promoters, 5′ UTR, 3′UTR, etc.) that results in a change in the level of ERAP1 expression, level of ERAP1 protein, and/or level of ERAP1 activity, is also expected to be useful to predict an increased or decreased likelihood of an AS patient responding to treatment with an IL-17 antagonist, e.g., secukinumab.

The presence or absence of an AS non-response allele, an AS response allele (or an AS risk marker), or an ERAP1 polymorphism that results in a change in the level or activity of ERAP1 (e.g., a decreased level of ERAP1 expression, level of ERAP1 protein or level of ERAP1 activity) may be detected by any of a variety of genotyping techniques. Typically, such genotyping techniques employ one or more oligonucleotides that are complementary to a region containing, or adjacent to, the polymorphic site (e.g., SNP) of interest. The sequence of an oligonucleotide used for genotyping a particular polymorphic site of interest is typically designed based on a context sequence or a reference sequence.

Numerous methods and devices are available to identify the presence or absence of an AS non-response allele, an AS response allele (or an AS risk marker), or an ERAP1 polymorphism that results in a decreased level of ERAP1 expression, level of ERAP1 protein or level of ERAP1 activity. DNA (genomic and cDNA) for SNP detection can be prepared from a biological sample by methods well known in the art, e.g., phenol/chloroform extraction, PUREGENE DNA® purification system from GentAS Systems (Qiagen, CA). Detection of a DNA sequence may include examining the nucleotide(s) located at either the sense or the anti-sense strand within that region. The presence or absence of polymorphisms may be detected from DNA (genomic or cDNA) obtained from PCR using sequence-specific probes, e.g., hydrolysis probes from Taqman, Beacons, Scorpions; or hybridization probes that detect the polymorphism. For the detection of the polymorphism, sequence specific probes may be designed such that they specifically hybridize to the genomic DNA for the alleles of interest or the cDNA for the sequence of interest. For example, sequence specific probes for rs27434 may be found in Li et al. (2011) J. Rheumatol. 38(2):317-21 and sequence specific probes for rs30187 may be found in Pazar et al. (2010) J. Rheumatol. 37(2):379-84. These probes may be labeled for direct detection or contacted by a second, detectable molecule that specifically binds to the probe. The PCR products also can be detected by DNA-binding agents. Said PCR products can then be subsequently sequenced by any DNA sequencing method available in the art. Alternatively the presence or absence of allele can be detected by sequencing using any sequencing methods such as, but not limited to, Sanger-based sequencing, pyrosequencing or next generation sequencing (Shendure J. and Ji, H., Nature Biotechnology (1998), Vol. 26, Nr 10, pages 1135-1145). In addition, optimised allelic discrimination assays for SNPs may be purchased from Applied Biosystems (Foster City, Calif., USA).

Various techniques can be applied to interrogate a particular polymorphism (e.g., SNP), including, e.g., hybridization-based methods, such as dynamic allele-specific hybridization (DASH) genotyping, polymorphic site (e.g., SNP) detection through molecular beacons (Abravaya K., et al. (2003) Clin Chem Lab Med. 41:468-474), Luminex xMAP technology, Illumina Golden Gate technology and commercially available high-density oligonucleotide SNP arrays (e.g., the Affymetrix Human SNP 5.0 GeneChip performs a genome-wide assay that can genotype over 500,000 human SNPs) BeadChip kits from Illumina, e.g., Human660W-Quad and Human 1.2M-Duo; enzyme-based methods, such as restriction fragment length polymorphism (RFLP), PCR-based methods (e.g., Tetra-primer ARMS-PCR), Invader assays (Olivier M. (2005) Mutat Res. 573(1-2):103-10), various primer extension assays (incorporated into detection formats, e.g., MALDI-TOF Mass spectrometry, electrophoresis, blotting, and ELISA-like methods), Taqman assays, and oligonucleotide ligase assays; and other post-amplification methods, e.g., analysis of single strand conformation polymorphism (Costabile et al. (2006) Hum. Mutat. 27(12):1163-73), temperature gradient gel electrophoresis (TGGE), denaturing high performance liquid chromatography, high-resolution melting analysis, DNA mismatch-binding protein assays (e.g., MutS protein from Thermus aquaticus binds different single nucleotide mismatches with different affinities and can be used in capillary electrophoresis to differentiate all six sets of mismatches), SNPLex® (proprietary SNP detecting system available from Applied Biosystems), capillary electrophoresis, mass spectrometry, and various sequencing methods, e.g. pyrosequencing and next generation sequencing, etc. Kits for SNP genotyping include Fluidigm Dynamic Array® IFCs (Fluidigm), TaqMan® SNP Genotyping Assay (Applied Biosystems), MassARRAY® iPLEX Gold (Sequenom), Type-it Fast® SNP Probe PCR Kit (Quiagen).

In some embodiments, the presence or absence of polymorphic site (e.g., SNP) in a patient is detected using a hybridization assay. In a hybridization assay, the presence or absence of the genetic marker is determined based on the ability of the nucleic acid from the sample to hybridize to a complementary nucleic acid molecule, e.g., an oligonucleotide probe. A variety of hybridization assays are available. In some, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide or binding agent (e.g., SYBR® Green), is allowed to contact the membrane under low-, medium- or high-stringency conditions. Unbound probe is removed and the presence of binding is detected by visualizing the labeled probe. In some embodiments, arrays, e.g., the MassARRAY system (Sequenom, San Diego, Calif., USA) may be used to genotype a subject.

Traditional genotyping methods (e.g., employed in HLA typing) may also be used for SNP genotyping and in identifying polymorphic sites (e.g., the SNPs of Table 1 and Table 2). Such traditional methods include, e.g., DNA amplification techniques such as PCR and variants thereof, direct sequencing, Sequence Specific Oligonucleotide (SSO) hybridization coupled with the Luminex xMAP® technology, Sequence Specific Primer (SSP) typing, and Sequence Based Typing (SBT). Sequence Based Typing (SBT) is based on PCR target amplification, followed by sequencing of the PCR products and data analysis. Sequence-Specific Oligonucleotide (SSO) typing uses PCR target amplification, hybridization of PCR products to a panel of immobilized sequence-specific oligonucleotides on the beads, detection of probe-bound amplified product by color formation followed by data analysis. Sequence Specific Primers (SSP) typing is a PCR based technique which uses sequence specific primers for DNA based typing. Skilled artisans will understand that genotyping with the described SSO, SBT and SSP typing may be performed using various commercially available kits from, e.g., Invitrogen.

In some cases, RNA, e.g., messenger RNA (mRNA—pre and/or mature) can also be used to determine the presence or absence of a polymorphic site (e.g., SNP). Analysis of the sequence of mRNA transcribed from a given gene can be performed using any known method in the art including, but not limited to, Northern blot analysis, nuclease protection assays (NPA), in situ hybridization, reverse transcription-polymerase chain reaction (RT-PCR), RT-PCR ELISA, TaqMan-based quantitative RT-PCR (probe-based quantitative RT-PCR) and SYBR green-based quantitative RT-PCR. In one example, detection of mRNA levels involves contacting the isolated mRNA with an oligonucleotide that can hybridize to mRNA encoded by an AI response marker. The nucleic acid probe can typically be, for example, a full-length cDNA, or a portion thereof, such as an oligonucleotide of at least 7, 15, 30, 50, or 100 nucleotides in length and sufficient to specifically hybridize under stringent conditions to the mRNA. Hybridization of an mRNA with the probe indicates that the marker in question is being expressed. In one format, the RNA is immobilized on a solid surface and contacted with a probe, for example by running the isolated RNA on an agarose gel and transferring the mRNA from the gel to a membrane, such as nitrocellulose. Amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers. PCR products can be detected by any suitable method including, but not limited to, gel electrophoresis and staining with a DNA-specific stain or hybridization to a labeled probe.

The level of expression of a gene (e.g., ERAP1) may be determined by measuring RNA (or reverse transcribed cDNA) levels using various well-known techniques, e.g., a PCR-based assay, reverse-transcriptase PCR (RT-PCR) assay, Northern blot, etc. Quantitative RT-PCR with standardized mixtures of competitive templates can also be utilized.

In some embodiments, the presence or absence of an AS non-response allele (in the case of the rs30187 non-response allele) or, in some cases, an AS risk marker, in a patient can be determined by analyzing polypeptide products (e.g., polypeptide products of the ERAP1 gene), in which non synonymous polymorphism can be derived from polypeptide sequence. Analysis of polypeptide products can be performed using any known method in the art including, but not limited, to immunocytochemical staining, protein sequencing, ELISA, flow cytometry, Western blot, spectrophotometry, HPLC, and mass spectrometry.

As described previously, we have determined that higher levels of AS response proteins (i.e., S100A8, S100A9, and S100A8+S100A9) associate with improved ASAS20 and ASAS40 response during secukinumab treatment. We thus contemplate that testing subjects for the level of at least one AS response protein will be useful in a variety of pharmacodiagnostic products and methods that involve identifying individuals more likely to respond to IL-17 antagonsim and in helping physicians decide whether to prescribe IL-17 antagonists (e.g., secukinumab) to a patient having AS.

We also conclude that modifications in the level or activity of ERAP1 (e.g., decreased levels of ERAP1 protein) may be predictive of an improved response to IL-17 antagonism (e.g., secukinumab treatment) for AS patients. We thus contemplate that testing subjects for the levels of ERAP1 protein will be useful in a variety of pharmacogenetic products and methods that involve identifying individuals more likely to respond to IL-17 antagonism and in helping physicians decide whether to prescribe IL-17 antagonists (e.g., secukinumab) to a patient having AS.

For detection of protein levels, there are numerous well-known method for detecting polypeptide products in a sample, e.g., by means of a probe (e.g., a binding protein, e.g., an antibody capable of interacting specifically with S100A8 and/or S100A9, an antibody capable of binding ERAP1). Labeled antibodies, binding portions thereof, or other binding partners can be used. The antibodies can be monoclonal or polyclonal in origin, or may be biosynthetically produced. The binding partners may also be naturally occurring molecules or synthetically produced. The amount of complexed proteins is determined using standard protein detection methodologies described in the art. A detailed review of immunological assay design, theory and protocols can be found in numerous texts in the art, including Practical Immunology, Butt, W. R., ed., Marcel Dekker, New York, 1984.

A variety of immunohistochemistry assays are available for detecting proteins with labeled antibodies. Direct labels include fluorescent or luminescent tags, metals, dyes, radionucleides, and the like, attached to the antibody. Indirect labels include various enzymes well known in the art, such as alkaline phosphatase, hydrogen peroxidase and the like. In a one-step assay, the target protein (e.g., ERAP1, S100A8, and/or S100A9) is immobilized and incubated with a labeled antibody. The labeled antibody binds to the immobilized target molecule. After washing to remove unbound molecules, the sample is assayed for the presence of the label. Numerous immunohistochemical methods are incorporated into point-of-care formats and hand-helds, all of which may be used for determining levels of protein.

The use of immobilized antibodies specific for the proteins or polypeptides is also contemplated by the present disclosure. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and processed through washes and detection steps to generate a measurable signal, e.g., a colored spot.

In a two-step assay, an immobilized target protein (e.g., ERAP1, S100A8, and/or S100A9) may be incubated with an unlabeled antibody. The unlabeled antibody complex, if present, is then bound to a second, labeled antibody that is specific for the unlabeled antibody. The sample is washed and assayed for the presence of the label. The choice of marker used to label the antibodies will vary depending upon the application. However, the choice of the marker is readily determinable to one skilled in the art.

The antibodies may be labeled with a radioactive atom, an enzyme, a chromophoric or fluorescent moiety, or a colorimetric tag. The choice of tagging label also will depend on the detection limitations desired. Enzyme assay's (ELISAs) typically allow detection of a colored product formed by interaction of the enzyme-tagged complex with an enzyme substrate. Some examples of radioactive atoms include ³²P, ¹²⁵I, ³H, and ¹⁴P. Some examples of enzymes include horseradish peroxidase, alkaline phosphatase, beta-galactosidase, and glucose-6-phosphate dehydrogenase. Some examples of chromophoric moieties include fluorescein and rhodamine. The antibodies may be conjugated to these labels by methods known in the art. For example, enzymes and chromophoric molecules may be conjugated to the antibodies by means of coupling agents, such as dialdehydes, carbodiimides, dimaleimides, and the like. Alternatively, conjugation may occur through a ligand-receptor pair. Some suitable ligand-receptor pairs include, e.g., biotin-avidin or -streptavidin, and antibody-antigen. ELISA-based kits for detecting calprotectin levels (S100A8/S100A9) may be purchased from Buhlmann Laboratroies AG, Schonenbuch Basel, Switzerland and PhiCal, Immundiagnostic AG, Bensheim, Germany.

In one aspect, the present disclosure contemplates the use of a sandwich technique for detecting the level of a target protein (e.g., ERAP1, S100A8, and/or S100A9) in biological samples. The technique requires two antibodies capable of binding the protein of interest: e.g., one immobilized onto a solid support and one free in solution, but labeled with some easily detectable chemical compound. Examples of chemical labels that may be used for the second antibody include but are not limited to radioisotopes, fluorescent compounds, and enzymes or other molecules which generate colored or electrochemically active products when exposed to a reactant or enzyme substrate. When samples containing an AS response protein are placed in this system, the polypeptide products binds to both the immobilized antibody and the labeled antibody. The result is a “sandwich” immune complex on the support's surface. The complexed protein is detected by washing away nonbound sample components and excess labeled antibody, and measuring the amount of labeled antibody complexed to protein on the support's surface. A sandwich immunoassay is highly specific and very sensitive when labels with good limits of detection are used.

Dot blotting is routinely practiced by the skilled artisan to detect a desired protein using an antibody as a probe (Promega Protocols and Applications Guide, Second Edition, 1991, Page 263, Promega Corporation). Samples are applied to a membrane using a dot blot apparatus. A labeled probe is incubated with the membrane, and the presence of the protein is detected.

Western blot analysis is well known to the skilled artisan (Sambrook et al., Molecular Cloning, A Laboratory Manual, 1989, Vol. 3, Chapter 18, Cold Spring Harbor Laboratory). In Western blot, the sample is separated by SDS-PAGE. The gel is transferred to a membrane. The membrane is incubated with labeled antibody for detection of the desired protein.

Mass spectrometry may also be used for detecting monomeric S100A8 and S100A9 and S100A8+S100A9 (de Seny et al. (2008) Clin. Chem. 54(6):1066-75; Tilleman et al. (2005) Proteomics 5(8):2247-57).

The assays described above involve steps such as, but not limited to, immunoblotting, immunodiffusion, immunoelectrophoresis, or immunoprecipitation. Specific immunological binding of the antibody to the protein or polypeptide can be detected directly or indirectly. In some embodiments, an automatic analyzer (e.g., a PCR machine or an automatic sequencing machine) is used to determine the level of an AS response protein, e.g., S100A8 and/or S100A9. All such methods are well known by skilled artisans. Preferably, the level of an AS response protein, e.g., S100A8 and/or S100A9, or ERAP1 in a sample is detected by radioimmunoassays or enzyme-linked immunoassays, competitive binding enzyme-linked immunoassays, mass spectrometry, point of care techniques/platforms, dot blot, Western blot, chromatography, preferably high performance liquid chromatography (HPLC), or other assays known in the art.

The level ERAP1 activity in a biological sample may be assayed by various methods disclosed in the art, e.g., via the methods set forth in Kochan et al. (2011) Proc Natl Acad Sci USA. 108(19):7745-50.

For comparative purposes, the level of ERAP1 expression, level of ERAP1 protein, or level of ERAP1 activity in a biological sample from a patient may be compared to the level of ERAP1 expression, level of ERAP1 protein, and level of ERAP1 activity from a control. The control may be a reference level of ERAP1 expression, level of ERAP1 protein, or level of ERAP1 activity derived from subjects (e.g., AS patients) known to respond well to treatment with an IL-17 antagonist (e.g., secukinumab) or subjects known to respond poorly to treatment with an IL-17 antagonist (e.g., secukinumab), as the case may be. A control level of expression may be derived from biological samples from reference subjects (i.e., AS patients known to respond well to treatment with an IL-17 antagonist (e.g., secukinumab) or AS patients known to respond poorly to treatment with an IL-17 antagonist (e.g., secukinumab)), or may simply be a numerical standard (e.g., mean, median, range, [+/− standard deviation]) previously derived from reference subjects. In some embodiments the control is a reference level of ERAP1 expression, level of ERAP1 protein, or level of ERAP1 activity derived from a subject known to respond poorly to treatment with an IL-17 antagonist and the level of ERAP1 expression, level of ERAP1 protein, or level of ERAP1 activity (as the case may be) from the patient is compared to this control, wherein a decreased level of ERAP1 expression, level of ERAP1 protein, or level of ERAP1 activity in the sample from the patient relative to the control provides an indication that the patient will have an increased likelihood of responding to treatment with the IL-17 antagonist (e.g., secukinumab). In other embodiments, the control is a reference level of ERAP1 expression, level of ERAP1 protein, or level of ERAP1 activity derived from a subject known to respond well to treatment with an IL-17 antagonist and the level of ERAP1 expression, level of ERAP1 protein, or level of ERAP1 activity from the patient to be treated is compared to this control, wherein a similar (e.g., statistically similar) level of ERAP1 expression, level of ERAP1 protein, or level of ERAP1 activity in the sample from the patient relative to the control provides an indication that the patient will have an increased likelihood of responding to treatment with the IL-17 antagonist (e.g., secukinumab).

An AS non-response allele, AS response allele, AS risk marker, or an ERAP1 polymorphism that results in modifications in the level and/or activity of ERAP1 (e.g., decreased level of ERAP1 expression, level of ERAP1 protein and/or level of ERAP1 activity) can also be identified by detecting an equivalent genetic marker thereof, which can be, e.g., another SNP (single nucleotide polymorphism), a microsatellite marker, another allele or other kinds of genetic polymorphisms. For example, the presence of a genetic marker on the same haplotype as an AS non-response allele or an ERAP1 polymorphism, rather than an AS non-response allele or an ERAP1 polymorphism per se, may be indicative of a patient's likelihood for responding to treatment with an IL-17 antagonist. Two particular alleles at different loci on the same chromosome are said to be in linkage disequilibrium (LD) if the presence of one of the alleles at one locus tends to predict the presence of the other allele at the other locus. Such variants, which are referred to herein as linked variants, or proxy variants, may be any type of variant (e.g., a SNP, insertion or deletion) that is in high LD with the better response allele of interest. The candidate linked variant may be an allele of a polymorphism that is currently known. Other candidate linked variants may be readily identified by the skilled artisan using any technique well-known in the art for discovering polymorphisms.

The degree of LD between alleles of interest and a candidate linked variant may be determined using any LD measurement known in the art. LD patterns in genomic regions are readily determined empirically in appropriately chosen samples using various techniques known in the art for determining whether any two alleles (e.g., between nucleotides at different PSs) are in linkage disequilibrium (see, e.g., GENETIC DATA ANALYSIS II, Weir, Sineuer Associates, Inc. Publishers, Sunderland, Mass. 1996). The skilled artisan may readily select which method of determining LD will be best suited for a particular population sample size and genomic region. One of the most frequently used measures of linkage disequilibrium is r, which is calculated using the formula described by Devlin et al. (Genomics, 29(2):311-22 (1995)). “r” is the measure of how well an allele X at a first locus predicts the occurrence of an allele Y at a second locus on the same chromosome. “r” only reaches 1.0 when the prediction is perfect.

Preferably, the locus of the linked variant is in a genomic region of about 200 kilobases, more preferably 100 kilobases, more preferably about 10 kb that spans one of the polymorphic sites disclosed in Tables 1 and 2. Other linked variants are those in which the LD with the better response allele has a r2 value, as measured in a suitable reference population, of at least 0.75, more preferably at least 0.80, even more preferably at least 0.85 or at least 0.90, yet more preferably at least 0.95, and most preferably 1.0. The reference population used for this r measurement may be the general population, a population using an IL-17 antagonist, a population diagnosed with a particular condition for which the IL-17 antagonists shows efficacy (such as an AS patient) or a population whose members are self-identified as belonging to the same ethnic group, such as Caucasian, African American, Hispanic, Latino, Native American and the like, or any combination of these categories. Preferably the reference population reflects the genetic diversity of the population of patients to be treated with the IL-17 antagonist.

Analysis of the level of AS response protein, level of ERAP1 expression, the level of ERAP1 protein, the level of ERAP1 activity, or presence (or absence) of an AS non-response allele, AS response allele, or AS response protein may be carried out separately or simultaneously while analyzing other genetic sequences (e.g., an AS risk marker). For example, a skilled artisan may analyze a sample for more than one AS non-response allele, more than one AS response allele, more than one AS risk marker, more than one AS response protein, and any combination thereof. Thus, in one aspect of the present disclosure, an array is provided to which probes that correspond in sequence to gene products, e.g., genomic DNA, cDNAs, mRNAs, cRNAs, polypeptides and fragments thereof, can be specifically hybridized or bound at a known position. As such, one may use such an array to concurrently analyze a biological sample from a patient for various genomic or biochemical markers of a patient.

In performing any of the methods described herein that require determining the presence of an AS non-response allele, an AS response allele, or ERAP1 polymorphism, the level of ERAP1 expression, the level of ERAP1 protein, or the level of ERAP1 activity such determination may be made by consulting a data repository that contains sufficient information on the patient's genetic composition to determine whether the patient has the marker of interest. Preferably, the data repository lists the genotype present (or absent) in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a Palm Pilot a tape, a zip disk, the computer's internal ROM (read-only-memory) or the internet or worldwide web. Other media for the storage of files accessible by a computer will be obvious to one skilled in the art.

Typically, once levels of ERAP1 expression, levels of ERAP1 protein/activity, levels of an AS response protein, the presence of an AS non-response allele, the presence of an AS response allele, or the presence of an ERAP1 polymorphisms are determined, physicians or genetic counselors or patients or other researchers may be informed of the result. Specifically the result can be cast in a transmittable form of information that can be communicated or transmitted to other researchers or physicians or genetic counselors or patients. Such a form can vary and can be tangible or intangible. The result can be embodied in descriptive statements, diagrams, photographs, charts, images or any other visual forms. For example, images of gel electrophoresis of PCR products can be used in explaining the results. Diagrams showing where a variant occurs in an individual's allele are also useful in indicating the testing results. Statements regarding levels of ERAP1 expression, levels of ERAP1 protein/activity, levels of an AS response protein, the presence of an AS non-response allele, the presence of an AS response allele, and the presence of an ERAP1 polymorphism are also useful in indicating the testing results. These statements and visual forms can be recorded on a tangible media such as papers, computer readable media such as floppy disks, compact disks, etc., or on an intangible media, e.g., an electronic media in the form of email or website on internet or intranet. In addition, the result can also be recorded in a sound form and transmitted through any suitable media, e.g., analog or digital cable lines, fiber optic cables, etc., via telephone, facsimile, wireless mobile phone, internet phone and the like. All such forms (tangible and intangible) would constitute a “transmittable form of information”. Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present disclosure also encompasses a method for producing a transmittable form of information containing data on levels of ERAP1 expression, levels of ERAP1 protein/activity, levels of an AS response protein, or the presence of an AS non-response allele, an AS response allele, or ERAP1 polymorphism in an individual. This form of information is useful for predicting the responsiveness of a patient having AS to treatment with an IL-17 antagonist, for selecting a course of treatment based upon that information, and for selectively treating a patient based upon that information.

Disclosed herein are methods of predicting the likelihood that a patient having AS will respond to treatment with an IL-17 antagonist (e.g., secukinumab), comprising detecting the presence of an AS non-response allele (an rs30187 non-response allele or an rs27434 non-response allele) or the presence of an AS response allele (an rs2201841 response allele or an rs11209032 response allele) in a biological sample from the patient, wherein: a) the presence of the AS non-response allele is indicative of a decreased likelihood that the patient will respond to treatment with the IL-17 antagonist; and b) the presence of the AS response allele is indicative of an increased likelihood that the patient will respond to treatment with the IL-17 antagonist. In some embodiments, the method further comprises the step of obtaining the biological sample from the patient, wherein the step of obtaining is performed prior to the step of detecting.

Disclosed herein are methods of predicting the likelihood that a patient having AS will respond to treatment with an IL-17 antagonist (e.g., secukinumab), comprising detecting a test level of at least one AS response protein (S100A8, S100A9, S100A8+S100A9) in a biological sample from the patient, wherein the patient has an increased likelihood of responding to treatment with the IL-17 antagonist if the test level is greater than a control level of the least one AS response protein and wherein the patient has a decreased likelihood of responding to treatment with the IL-17 antagonist if the test level is less than the control level.

Disclosed herein are methods of predicting the likelihood that a patient having AS will respond to treatment with an IL-17 antagonist (e.g., secukinumab), comprising: a) detecting a test level of at least one AS response protein (S100A8, S100A9, S100A8+S100A9) in a biological sample from the patient; and b) comparing the test level of the at least one AS response protein to a control level of the at least one AS response protein, wherein the patient has an increased likelihood of responding to treatment with the IL-17 antagonist if the test level is greater than the control level and wherein the patient has a decreased likelihood of responding to treatment with the IL-17 antagonist if the test level is less than the control level.

In some embodiments, the detecting step is performed by directly assaying the biological sample from the patient for the subject matter (e.g., allele, protein level, etc.) of interest. In some embodiments of the above methods, the presence or absence of an allele of interest may be detected by assaying the biological sample for a genomic sequence, a nucleic acid product, a polypeptide product, or an equivalent genetic marker. The allele of interest may be the rs30187 non-response allele, the rs27434 non-response allele, the rs2201841 response allele, and/or the rs11209032 response allele. In some embodiments of the above methods, the biological sample is additionally assayed for the presence of at least one AS risk marker selected from the group consisting of an IL-23R SNP, an IL-1R2 SNP, an ANTXR2 SNP, an IL-17A SNP and an HLA allele, e.g., an rs11209026 allele, an rs10865331 allele, an rs2310173 allele, an rs4333130 allele, an rs2242944 allele, an rs1974226 allele, an rs7747909 allele, HLA-DRB1*04 or HLA-B*27.

Disclosed herein are also various methods of predicting the likelihood that a patient having AS will respond to treatment with an IL-17 antagonist (e.g., secukinumab), comprising detecting the level of ERAP1 expression (e.g., mRNA, cDNA, etc.), the level of ERAP1 protein, or the level of ERAP1 activity in a biological sample from the patient relative to a control; wherein a decreased level of ERAP1 expression, decreased level of ERAP1 protein, or a decreased level of ERAP1 activity relative to the control is indicative of an increased likelihood that the patient will respond to treatment with the IL-17 antagonist (e.g., secukinumab). In such an embodiment, the control is a reference level of ERAP1 expression, level of ERAP1 protein, or level of ERAP1 activity derived from a subject known to respond poorly to treatment with an IL-17 antagonist.

Disclosed herein are also various methods of predicting the likelihood that a patient having AS will respond to treatment with an IL-17 antagonist (e.g., secukinumab), comprising detecting the level of ERAP1 expression (e.g., mRNA, cDNA, etc.), the level of ERAP1 protein, and/or the level of ERAP1 activity in a biological sample from the patient relative to a control; wherein a similar level of ERAP1 expression, similar level of ERAP1 protein, or a similar level of ERAP1 activity relative to the control is indicative of an increased likelihood that the patient will respond to treatment with the IL-17 antagonist (e.g., secukinumab). In such an embodiment, the control is a reference level of ERAP1 expression, level of ERAP1 protein, or level of ERAP1 activity derived from a subject known to respond well to treatment with an IL-17 antagonist.

In some embodiments, the level of ERAP1 expression, the level of ERAP1 protein, or the level of ERAP1 activity is measured by assaying the biological sample for an ERAP1 polymorphism that results in a decreased level of ERAP1 expression, a decreased level of ERAP1 protein, and/or a decreased level of ERAP1 activity relative to the control.

In some embodiments of the above methods, the biological sample is selected from the group consisting of synovial fluid, blood, serum, feces, plasma, urine, tear, hair bulb cells, saliva, cerebrospinal fluid, a leukocyte sample and a tissue sample. In some embodiments of the above methods, the presence or absence of the allele of interest or the level of the protein of interest (as the case may be) is detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, Western Blot, protein sequencing, immunoassays, immunohistochemistry, ELISA, flow cytometry, HPLC, and mass spectrometry. Assaying may be performed by use of an “automatic analyzer”, which is any machine that can be used to determine the presence or absence of an allele of interest. For example, a PCR machine, automatic sequencer, spectrometer, densitometer, plate reader, scintillation counter, etc.

In some embodiments of the above methods, the alleles of interest are used to prodict either short or long term outcome. Short term outcome may be measured by, e.g., improvement in the signs and symptoms of inflammation, while long term outcomes can be measured by, e.g., conventional radiographs and MRI as imaging surrogates, functional scores (BASFI, BASMI) and quality of life (QoL) tools.

### Methods of Treatment and Uses of IL-17 Antagonists

The disclosed methods allow clinicians to provide a personalized therapy for AS patients, i.e., they allow determination of whether to treat an AS patient with an IL-17 antagonist or whether to treat the AS patient with a different AS agent (e.g., an NSAID, TNF alpha antagonist, DMARD, corticosteroid, or combinations thereof). In this way, a clinician can maximize the benefit and minimize the risk of IL-17 antagonism in the entire population of patients afflicted with AS. It will be understood that IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) are useful for the treatment, prevention, or amelioration of AS (e.g., signs and symptoms of inflammation and clinical and imaging evidence of structural changes, preventing further joint erosion, improving joint structure, preventing ankylosis, preventing long-term disability, etc. [e.g., improvement in BASFI, BASMI, enthesitis scores, quality of life (QoL) scores, etc.]), particularly in AS patients that do not have an AS non-response allele, that have an AS response allele or who have elevated levels of one or several AS response proteins.

The IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof), may be used in vitro, ex vivo, or incorporated into pharmaceutical compositions and administered to individuals (e.g., human patients) in vivo to treat, ameliorate, or prevent AS, e.g., in AS patients that do not have an AS non-response allele, who have an AS response allele, who have elevated levels of an AS response protein, or who have decreased levels of ERAP1 expression and/or levels of ERAP1 protein/activity. A pharmaceutical composition will be formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration.

The IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof).

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise liquid or lyophilized formulation.

Antibodies, e.g., antibodies to IL-17, are typically formulated either in aqueous form ready for parenteral administration or as lyophilisates for reconstitution with a suitable diluent prior to administration. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are well known in the art. When a therapeutically effective amount of an IL-17 antagonist, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's, dextrose, dextrose and sodium chloride, lactated Ringer's, or other vehicle as known in the art.

The appropriate dosage will, of course, vary depending upon, for example, the particular IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) to be employed, the host, the mode of administration and the nature and severity of the condition being treated, and on the nature of prior treatments that the patient has undergone. Ultimately, the attending health care provider will decide the amount of the IL-17 antagonist with which to treat each individual patient. In some embodiments, the attending health care provider may administer low doses of the IL-17 antagonist and observe the patient's response. In other embodiments, the initial dose(s) of IL-17 antagonist administered to a patient are high, and then are titrated downward until signs of relapse occur. Larger doses of the IL-17 antagonist may be administered until the optimal therapeutic effect is obtained for the patient, and the dosage is not generally increased further.

In practicing some of the methods of treatment or uses of the present disclosure, a therapeutically effective amount of an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) is administered to a patient, e.g., a mammal (e.g., a human). While it is understood that the disclosed methods provide for differential treatment of AS patients depending on the presence (or absence) of AS non-response alleles, AS response alleles and the levels of AS response proteins, this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered either alone or in combination with other agents and therapies for treating AS patients, e.g., in combination with at least one additional agent, such as an immunosuppressive agent, a disease-modifying anti-rheumatic drug (DMARD) (e.g., sulfasalazine), a pain-control drug, a steroid, a non-steroidal anti-inflammatory drug (NSAID), a cytokine antagonist, a bone anabolic, a bone anti-resorptive, and combinations thereof (e.g., dual and tripple therapies). When coadministered with one or more additional AS agents, an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery.

Non-steroidal anti inflammatory drugs and pain control agents useful in combination with an IL-17 antagonist (e.g., secukinumab) for the treatment of AS patients include, propionic acid derivative, acetic acid derivative, enolic acid derivatives, fenamic acid derivatives, Cox inhibitors, e.g., lumiracoxib, ibuprophen, fenoprofen, ketoprofen, flurbiprofen, oxaprozin, indomethacin, sulindac, etodolac, ketorolac, nabumetone, aspirin, naproxen, valdecoxib, etoricoxib, MK0966, rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib. DMARDs useful in combination with an IL-17 antagonist, e.g., secukinumab, for the treatment of AS patients that do not have an AS non-response allele include, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil. Steroids (e.g., glucocorticoids) useful in combination with an IL-17 antagonist, e.g., secukinumab, for the treatment of AS patient that do not have an AS non-response allele include, Prednisolone, Prednisone, dexamethasone, cortisol, cortisone, hydrocortisone, methylprednisolone, betamethasone, triamcinolone, beclometasome, fludrocottisone, deoxycorticosterone, aldosterone.

Biologic agents potentially useful in combination with an IL-17 antagonist, e.g., secukinumab, for the treatment of AS patients include, ADALIMUMAB (Humira®), ETANERCEPT (Enbrel®), INFLIXIMAB (Remicade®; TA-650), ILLARIS® (canakinumab), CERTOLIZUMAB PEGOL (Cimzia®; CDP870), GOLIMUMAB (Simponi®; CNTO148), ANAKINRA (Kineret®), RITUXIMAB (Rituxan®; MabThera®), ABATACEPT (Orencia®), TOCILIZUMAB (RoActemAS/Actemra®), integrin antagonists (TYSABRI® (natalizumab)), CD4 antagonists, further IL-17 antagonists (LY2439821, RG4934, AMG827, SCH900117, R05310074, MEDI-571, CAT-2200), IL-23 antagonists, IL-20 antagonists, IL-6 antagonists, TNF alpha antagonists (e.g., TNF alpha antagonists or TNF alpha receptor antagonists, e.g., pegsunercept, etc.), BLyS antagonists (e.g., Atacicept, Benlysta®/LymphoStat-B® (belimumab)), p38 Inhibitors, CD20 antagonists (Ocrelizumab, Ofatumumab (Arzerra®)), Interferon gamma antagonists (Fontolizumab).

An IL-17 antagonist, e.g., secukinumab, is conveniently administered parenterally, intravenously, e.g., into the antecubital or other peripheral vein, intramuscularly, or subcutaneously. The duration of intravenous (i.v.) therapy using a pharmaceutical composition of the present disclosure will vary, depending on the severity of the disease being treated and the condition and personal response of each individual patient. Also contemplated is subcutaneous (s.c.) therapy using a pharmaceutical composition of the present disclosure. The health care provider will decide on the appropriate duration of i.v. or s.c. therapy and the timing of administration of the therapy, using the pharmaceutical composition of the present disclosure.

Preferred dosing and treatment regimens (including both induction and maintenance regimens) for treating AS patients that do not have an AS non-response allele, that have an AS response allele or who have elevated levels of an AS response protein are provided in PCT Application No. PCT/US2011/064307, which is incorporated by reference herein in its entirety). It will be understood that dose escalation may be required (e.g., during an induction and/or maintenance phase) for certain patients, e.g., patients that display inadequate response to treatment with the IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof). Thus, s.c. dosages of secukinumab may be greater than about 75 mg to about 300 mg s.c., e.g., about 80 mg, about 100 mg, about 125 mg, about 175 mg, about 200 mg, about 250 mg, about 350 mg, about 400 mg, etc.; similarly, i.v. dosages may be greater than about 10 mg/kg, e.g., about 11 mg/kg, 12 mg/kg, 15 mg/kg, 20 mg/kg, 25 mg/kg, 30 mg/kg, 35 mg/kg, etc. It will also be understood that dose reduction may also be required (e.g., during an induction and/or maintenance phase) for certain patients, e.g., patients that display an adverse response to treatment with the IL-17 antagonist (e.g., secukinumab). Thus, dosages of secukinumab may be less than about 75 mg to about 300 mg s.c., e.g., about 25 mg, about 50 mg, about 80 mg, about 100 mg, about 125 mg, about 175 mg, about 200 mg, 250 mg, etc.; similarly, i.v. dosages may be less than about 10 mg/kg, e.g., about 9 mg/kg, 8 mg/kg, 5 mg/kg, 4 mg/kg, 3 mg/kg, 2 mg/kg, 1 mg/kg etc.

Disclosed herein are methods of selectively treating a patient having AS, comprising selectively administering a therapeutically effective amount of an IL-17 antagonist to the patient on the basis of said patient having an AS response allele selected from an rs2201841 response allele or an rs11209032 response allele. Disclosed herein are also methods of selectively treating a patient having AS, comprising selectively administering a therapeutically effective amount of an IL-17 antagonist to the patient on the basis of said patient not having an AS non-response allele selected from an rs30187 non-response allele or an rs27434 non-response allele.

Disclosed herein are methods of selectively treating a patient having AS, comprising either: a) selectively administering a therapeutically effective amount of an IL-17 antagonist (e.g., secukinumab) to the patient on the basis of said patient having an AS response allele (rs2201841 response allele or an rs11209032 response allele) or on the basis of said patient not having an AS non-response allele (an rs30187 non-response allele or an rs27434 non-response allele); or b) selectively administering a therapeutically effective amount of a different AS agent (e.g., an NSAID, a TNF alpha antagonist, sulfasalazine, methotrexate, a corticosteroid and combinations thereof) to the patient on the basis of said patient not having an AS response allele or on the basis of said patient having an AS non-response allele.

Disclosed herein are also methods of selectively treating a patient having AS with an IL-17 antagonist, comprising: a) selecting the patient for treatment with the IL-17 antagonist (e.g., secukinumab) on the basis of a the patient having an AS response allele (an rs2201841 response allele or an rs11209032 response allele) or on the basis of the patient not having an AS non-response allele (an rs30187 non-response allele or an rs27434 non-response allele); and b) thereafter, administering a therapeutically effective amount of the IL-17 antagonist to the patient.

Disclosed herein are also methods of selectively treating a patient having AS with an IL-17 antagonist (e.g., secukinumab), comprising: a) assaying a biological sample from the patient for the presence or absence of an AS response allele (an rs2201841 response allele or an rs11209032 response allele) or an AS non-response allele (an rs30187 non-response allele or an rs27434 non-response allele); and b) thereafter, selectively administering to the patient either: i. a therapeutically effective amount of an IL-17 antagonist to the patient on the basis of the biological sample from the patient having an AS response allele or on the basis of the biological sample from the patient not having an AS non-response allele; or ii. a therapeutically effective amount of a different AS agent (e.g., an NSAID, a TNF alpha antagonist, sulfasalazine, methotrexate, a corticosteroid and combinations thereof) on the basis of the biological sample from the patient not having an AS response allele or on the basis of the biological sample from the patient having an AS non-response allele.

Disclosed herein are also methods of selectively treating a patient having AS with an IL-17 antagonist (e.g., secukinumab), comprising: a) assaying a biological sample from the patient for the presence or absence of an AS response allele (an rs2201841 response allele or an rs11209032 response allele) or an AS non-response (an rs30187 non-response allele or an rs27434 non-response allele) allele; b) thereafter, selecting the patient for treatment with the IL-17 antagonist on the basis of the biological sample from the patient having the AS response allele or on the basis of the biological sample from the patient not having the AS non-response allele; and c) thereafter, administering a therapeutically effective amount of the IL-17 antagonist to the patient.

In some embodiments, the method further comprises the step of assaying the biological sample from the patient for a test level of at least one AS response protein, which is performed prior to the step of administering.

In some embodiments, the AS non-response allele or the AS response allele is detected by assaying the biological sample for a genomic sequence of the AS non-response allele or the AS response allele, a nucleic acid product of the AS non-response allele or the AS response allele, a polypeptide product of the AS non-response allele or the AS response allele, or an equivalent genetic marker of the AS non-response allele or the AS response allele. In some embodiments, the presence of the at least one AS non-response allele or the presence of the AS response allele is detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, immunoassays, immunohistochemistry, ELISA, flow cytometry, Western blot, HPLC, and mass spectrometry.

Disclosed herein are methods of selectively treating a patient having AS, comprising either: a) selectively administering a therapeutically effective amount of an IL-17 antagonist (e.g., secukinumab) to the patient on the basis of said patient having a test level of at least one AS response protein (S100A8, S100A9, S100A8+S100A9) that is greater than a control level of the least one AS response protein; or b) selectively administering a therapeutically effective amount of a different AS agent (e.g., NSAID, a TNF antagonist, sulfasalazine, methotrexate, a corticosteroid and combinations thereof) to the patient on the basis of said patient having a test level of the least one AS response protein that is less than a control level of the least one AS response protein.

Disclosed herein are methods of selectively treating a patient having AS with an IL-17 antagonist, comprising: a) selecting the patient for treatment with the IL-17 antagonist (e.g., secukinumab) on the basis of said patient having a test level of at least one AS response protein (S100A8, S100A9, S100A8+S100A9) that is greater than a control level of the least one AS response protein; and b) thereafter, administering a therapeutically effective amount of the IL-17 antagonist to the patient.

Disclosed herein are methods of selectively treating a patient having AS, comprising: a) assaying a biological sample from an AS patient for a test level of at least one AS response protein (S100A8, S100A9, S100A8+S100A9); and b) thereafter selectively administering to the patient either: i. a therapeutically effective amount of an IL-17 antagonist (e.g., secukinumab) on the basis of the test level of the at least one AS response protein being greater than a control level of the least one AS response protein; or ii. a therapeutically effective amount of a different AS agent on the basis of the test level of the least one AS response protein being less than a control level of the least one AS response protein.

Disclosed herein are methods of selectively treating a patient having AS with an IL-17 antagonist (e.g., secukinumab), comprising: a) assaying a biological sample from an AS patient for a test level of at least one AS response protein (S100A8, S100A9, S100A8+S100A9); b) thereafter, selecting the patient for treatment with the IL-17 antagonist on the basis of the test level of the at least one AS response protein being greater than a control level of the least one AS response protein; and c) thereafter, administering a therapeutically effective amount of the IL-17 antagonist to the patient.

In some embodiments, the test level of the AS response protein is detected by a technique selected from the group consisting of immunoassay, immunohistochemistry, ELISA, Western blot, HPLC, and mass spectrometry. In some embodiments, the biological sample is additionally assayed for the presence of an AS non-response allele, an AS response allele, an AS risk marker, or combinations thereof.

In some embodiments, the control level is derived from a predetermined reference standard or a control biological sample from an IL-17 non-responder. In some embodiments, the test level is derived from analyzing the level of a polypeptide product of the at least one AS risk protein.

Disclosed herein is an IL-17 antagonist for use in the manufacture of a medicament for use in treating a patient having AS, wherein the patient is selected for treatment on the basis of not having an AS non-response allele or wherein the patient is selected for treatment on the basis of having an AS response allele.

Disclosed herein is an IL-17 antagonist for the manufacture of a medicament for the treatment of AS in a patient characterized as not having an AS non-response allele or a patient characterized as having an AS response allele, wherein the medicament is formulated to comprise containers, each container having either: a sufficient amount of the IL-17 antagonist to allow delivery of at least about 75 mg-about 150 mg of the IL-17 antagonist per unit dose; or a sufficient amount of the IL-17 antagonist to allow delivery of at least about 10 mg of the IL-17 antagonist/kg patient weight per unit dose. Also disclosed herein is an IL-17 antagonist for the manufacture of a medicament for the treatment of AS in a patient characterized as not having an AS non-response allele or a patient characterized as having an AS response allele, wherein the medicament is formulated at a dosage to allow either: intravenous delivery of about 10 mg of the IL-17 antagonist/kg patient weight per unit dose; or subcutaneous delivery of about 75 mg-about 150 mg of the IL-17 antagonist per unit dose.

Disclosed herein is an in vitro test method for selecting a patient for treatment of AS using an IL-17 antagonist, comprising determining if the patient has no AS non-response allele or determining if the patient has at least one AS response allele, wherein the patient has an improved therapeutic response to the following regimen: a) administering the patient three doses of about 10 mg/kg of an IL-17 antagonist, each of said doses being delivered every other week; and a) thereafter administering the patient about 75 mg-about 300 mg of the IL-17 antagonist twice a month, monthly, every two months or every three months, beginning during week eight.

Disclosed herein is an in vitro test method for selecting a patient for treatment of AS using an IL-17 antagonist, comprising determining if the patient has no AS non-response allele or determining if the patient has at least one AS response allele, wherein the patient has an improved therapeutic response to the following regimen: a) administering the patient five doses of about 75 mg-about 300 mg of an IL-17 antagonist, each of said doses being delivered weekly; and b) thereafter administering the patient about 75 mg-about 300 mg of the IL-17 antagonist twice a month, monthly, every two months or every three months, beginning during week eight.

Disclosed herein are also methods of treating an AS patient, comprising receiving data regarding the presence of an AS non-response allele or the presence of an AS response allele in a biological sample obtained from said patient; and selectively administering a therapeutically effective amount of an IL-17 antagonist to the AS patient if the patient does not have the AS non-response allele or administering a therapeutically effective amount of an IL-17 antagonist to the AS patient if the patient has the AS response allele. The phrase “receiving data” is used to mean obtaining possession of information by any available means, e.g., orally, electronically (e.g., by electronic mail, encoded on diskette or other media), written, etc.

Disclosed herein are various methods of selectively treating a patient having AS, comprising assaying a biological sample from the patient for the level of ERAP1 expression (e.g., mRNA, cDNA, etc.), the level of ERAP1 protein, or the level of ERAP1 activity; and thereafter selectively administering a therapeutically effective amount of an IL-17 antagonist, e.g., secukinumab, to the patient if the patient has a decreased ERAP1 expression, decreased level of ERAP1 protein, or a decreased level of ERAP1 activity relative to a control. In such an embodiment, the control is a reference level of ERAP1 expression, level of ERAP1 protein, or level of ERAP1 activity derived from a subject known to respond poorly to treatment with an IL-17 antagonist.

In some embodiments, the level of ERAP1 expression, the level of ERAP1 protein, or the level of ERAP1 activity is measured by assaying the biological sample from the patient for an ERAP1 polymorphism that results in a decreased level of ERAP1 expression, a decreased level of ERAP1 protein, or a decreased level of ERAP1 activity relative to the control.

Some of the above methods further comprise the step of obtaining the biological sample from the patient prior to the assaying step.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 75 mg, then a clinician may use 3 ml from a container that contains an IL-17 antibody formulation with a concentration of 25 mg/ml, 2 ml from a container that contains an IL-17 antibody formulation with a concentration of 37.5 mg/ml, 1 ml from a container that contains an IL-17 antibody formulation with a concentration of 75 mg/ml, 0.5 ml from a container contains an IL-17 antibody formulation with a concentration of 150 mg/ml, etc. In each such case, these containers have a sufficient amount of the IL-17 antagonist to allow delivery of the desired 75 mg dose.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., s.c. or i.v.). As an example, if a desired subcutaneous dose is 75 mg, then a clinician may use 2 ml of an IL-17 antibody formulation having a concentration of 37.5 mg/ml, 1 ml of an IL-17 antibody formulation having a concentration of 75 mg/ml, 0.5 ml of an IL-17 antibody formulation having a concentration of 150 mg/ml, etc. In each such case, these IL-17 antibody formulations are at a concentration high enough to allow subcutaneous delivery of the IL-17 antibody. Subcutaneous delivery typically requires delivery of volumes of less than about 2 ml, preferably a volume of about 1 ml or less.

### Kits

The invention also encompasses kits for detecting an AS non-response allele, an AS response allele, an ERAP1 polymorphism, an AS response protein, an ERAP1 protein, or level of ERAP1 activity in a biological sample from a patient. Such kits can be used to predict if a patient having AS is likely to respond (or have a higher response) to treatment with an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof). For example, the kit can comprise a probe (e.g., an oligonucleotode, antibody, labeled compound or other agent) capable of detecting the presence (or absence) of an AS non-response allele, an AS response allele, and/or an ERAP1 polymorphism that results in modification in the level and/or activity of ERAP1 (e.g., causes a decreased level of expression of ERAP1, a decreased level of ERAP1 protein, or a decreased level of ERAP1 activity), products of those alleles and/or an equivalent genetic marker of those alleles in a biological sample. The kit may also comprise instructions for providing a prediction of the likelihood that the patient will respond to treatment with the IL-17 antagonist.

Probes may specifically hybridize to genomic sequences, nucleic acid products, or polypeptide products. Exemplary probes are oligonucleotides or conjugated oligonucleotides that specifically hybridizes to the rs2201841, rs11209032, rs30187 or rs27434 polymorphic sites; a PCR primer, together with another primer, for amplifying the rs2201841, rs11209032, rs30187 or rs27434 polymorphic sites (e.g., from DNA, cDNA, mRNA, etc.); an antibody that is capable of differentiating between polypeptide products encoded by the disclosed alleles (e.g., an antibody that is capable of differentiating between a Lys528 and an Arg528 in an ERAP1 protein), an antibody capable of detecting S100A8, S100A9, or S100A8+S100A9, primer-extension oligonucleotides, allele-specific primers, a combination of allele-specific primers, allele-specific probes, and primer extension primers, etc. Optionally, the kit can contain a probe that targets an internal control allele, which can be any allele presented in the general population. Detection of an internal control allele is designed to assure the performance of the kit. The disclosed kits can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for use.

Such kits may also comprise an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) (e.g., in liquid or lyophilized form) or a pharmaceutical composition comprising the IL-17 antagonist (described supra). Additionally, such kits may comprise means for administering the IL-17 antagonist (e.g., a syringe and vial, a prefilled syringe, a prefilled pen) and instructions for use. These kits may contain additional therapeutic agents (described supra) for treating AS, e.g., for delivery in combination with the enclosed IL-17 antagonist, e.g., secukinumab.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, etc. With such items, a patient may self-administer the drug (i.e., administer the drug on their own behalf) or a physician may administer the drug.

The disclosed kits may be multiplex kits useful for simultaneously measuring the presence or absence of genetic components (e.g., particular SNPs) and the presence or absence of proteins or the levels thereof, which will allow physicians to make composite marker predictions.

### General

It will be understood that, in the above-mentioned methods, therapeutic regimens, kits, uses, and pharmaceutical compositions, an artisan may analyze more than one allele or marker. It is envisioned that a composite maker panel may be used for determining treatment decisions or predicting patient response, e.g., including analysis of one or several proteins, one or several SNPs, or combinations thereof (i.e., proteins and SNPs). For example, it is envisioned that a clinician may choose to analyze one or more ERAP1 SNPs, one or more IL23R SNPs and levels of S100A8 and/or S100A9 in a single patient. In some embodiments, even further combinations of biomarkers are analyzed, e.g., additional genetic markers (AS risk markers), transcription markers (e.g., mRNA/miRNA derived form blood, PBMCs, biopsies, etc.), and protein and cellular markers (e.g., protein biomarkers in serum and Th17 and Treg cells).

In some embodiments of the disclosed methods, treatments, regimens, uses and kits, the IL-17 binding molecule is selected from the group consisting of: a) an IL-17 antibody that binds to an epitope of IL-17 comprising Leu74, Tyr85, His86, Met87, Asn88, Val124, Thr125, Pro126, Ile127, Val128, His129; b) an IL-17 antibody that binds to an epitope of IL-17 comprising Tyr43, Tyr44, Arg46, Ala79, Asp80; c) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 binding molecule has a K_{D} of about 100-200 pM, and wherein the IL-17 binding molecule has an in vivo half-life of about 23 to about 35 days; and e) an IL-17 antibody that comprises an antibody selected from the group consisting of: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth PsA SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; and viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6. In preferred embodiments of the disclosed methods, treatments, regimens, uses and kits, the IL-17 antagonist is secukinumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed patient matter, as defined by the appended claims.

### EXAMPLES

### Example 1

### Proof of Concept AS Trial CAIN457A2209

### Example 1.1

### Study Design CAIN457A2209

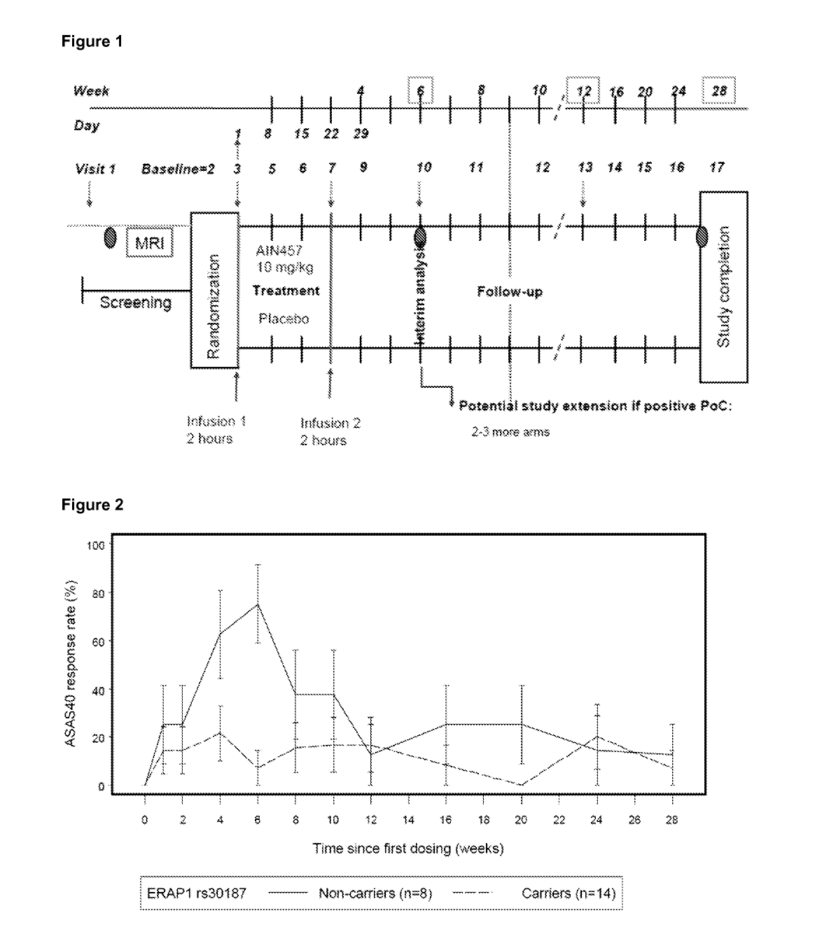

This was a two-part multi-center proof of concept study of multiple 10 mg/kg, 1.0 mg/kg and 0.1 mg/kg doses of secukinumab (2 infusions given 3 weeks apart) for the treatment of patients with a diagnosis of moderate to severe AS with or without previous TNF antagonist therapy (FIG. 1). In Part 1, 30 patients received either secukinumab 10 mg/kg or placebo in a 4:1 ratio. In Part 2, a further 30 patients received either secukinumab 0.1 mg/kg, 1.0 mg/kg or 10 mg/kg in a 2:2:1 ratio. The study consisted of a screening period of 28 days; a treatment period of 3 weeks, and a follow-up period of 25 weeks. Subjects who met the inclusion/exclusion criteria at screening underwent baseline evaluations, including the ASAS core set domains (1-6) (Zochling et al (2006) Ann Rheum Dis 65:442-452), BASMI score, BASDAI score and physician global assessment. The primary end point for this trial was the proportion of patients achieving the ASAS20 response at week 6.

Patients with moderate to severe AS fulfilling the modified New York criteria for a diagnosis of AS and whose disease was not controlled on NSAIDS (on at least one NSAID over a period of at least 3 months at maximum dose) were randomized to receive 2×10 mg/kg AIN457 or placebo. Minimum disease activity for inclusion of patients was assessed based on the ASAS core set domains: back pain & nocturnal pain score 4 despite concurrent NSAID use, PLUS a BASDAI score ≧4. Concomitant use of stable doses of methotrexate (MTX), sulphasalazine (SSZ) and low-dose corticosteroids was allowed as defined in the inclusion/exclusion criteria. Immunosuppressive agents other than MTX, SSZ and systemic low-dose corticosteroids required a 1-month wash-out period prior to baseline.

Efficacy evaluations were based on the ASAS core set and consist of the following assessment domains: (1) patient global assessment (PGA), (2) inflammatory back pain, (3) Bath Ankylosing Spondylitis Functional Index (BASFI), (4) morning stiffness by Bath Ankylosing Spondylitis Disease Activity Index (BASDAI). Secondary objectives included magnetic resonance imaging (MRI) studies of the spine using a scoring system for quantification of AS-related pathologies, to investigate whether these changes are affected by treatment with AIN457. Exploratory goals of the study were to define biomarker profiles using genetic, mRNA expression profiling, flow cytometry, and serum protein assessments in patients with moderate to severe AS, and to determine whether treatment with secukinumab affects these biomarkers.

Thirty (30) patients were randomized in a 4:1 ratio to receive two i.v infusions of either secukinumab (AIN457) 10 mg/kg i.v. or placebo i.v. given 3 weeks apart (Day 1 and Day 22). Patients were followed for safety up to week 28. A Bayesian analysis of the Week 6 ASAS20 response rates of AIN457 and placebo was performed. The prior distributions for the response rates were specified as Beta distributions and the binomial distribution was assumed for the observed number of responders in each group. The predictive distribution of the placebo response rate from a meta-analysis of 8 randomized, placebo-controlled trials of anti-TNFalpha treatment in AS was used as the prior distribution for the placebo response rate. This prior was equivalent to observing 11 out of 43 responders (i.e. a response rate of 26%). A weak prior was used for the active response rate (equivalent to observing 0.5 out of 1.5 responders). Sagittal MR images of the spine were performed including T1- and short tau inversion recovery (STIR) sequences at baseline, week 6 and week 28. Images were analyzed by an independent reader, who was blinded to treatment allocation and chronology of images, using the “Berlin modification” of the AS spinal MRI (ASspiMRI-a(2)) scoring system. Wilcoxon signed-rank test was used for the evaluation of changes between baseline and follow-up in each treatment arm.

### ASAS (Assessment in SpondyloArthritis)

The ASAS (Assessment in SpondyloArthritis) core set (1-6) consists of the following assessment domains: (1) Patient global assessment of disease activity, assessed on a 100 mm visual analogue scale (VAS); (2) Inflammatory back pain, assessed on a 100 mm VAS; (3) Physical function, assessed by BASFI; (4) Morning stiffness (spinal mobility), assessed using the BASDAI; (5) Bath Ankylosing Spondylitis Metrology Index (BASMI) scores (cervical rotation, chest expansion, lumbar lateral flexion, modified Schober index, occiput-to-wall distance); (6) Maastricht Ankylosing Spondylitis Enthesitis Score (MASES) and Leeds enthesis index.

ASAS20 Responder Definition

A subject is defined as an ASAS20 responder if, and only if, both of the following conditions hold:

1. they have a ≧20% improvement and an absolute improvement ≧1 unit in 3 of the following 4 domains: Patient Global Assessment (measured on a VAS scale, 0-10); Pain (measured on a VAS scale, 0-10); Function (as measured by the BASFI, 0-10); Inflammation (as measured by the mean of the two morning stiffness related questions from the BASDAI, 0-10);

2. they have no deterioration in the potential remaining domain (deterioration is defined as ≧20% worsening and an absolute worsening of ≧1 unit)

ASAS40 Responder Definition

A subject is defined as an ASAS40 responder if, and only if, both of the following conditions hold:

1. they have ≧40% improvement and an absolute improvement ≧2 units in 3 of the following 4 domains: Patient Global Assessment (measured on a VAS scale, 0-10); Pain (measured on a VAS scale, 0-10); Function (as measured by the BASFI, 0-10); Inflammation (as measured by the mean of the two morning stiffness related questions from the BASDAI, 0-10);

2. they have no deterioration in the potential remaining domain (deterioration is defined as ≧40% worsening and an absolute worsening of ≧2 units)

ASAS 5/6 Responder Definition

A subject is defined as an ASAS 5/6 responder if, and only if, they have at least a 20% improvement in five out of the following six domains: Patient Global Assessment (measured on a VAS scale, 0-10); Pain (measured on a VAS scale, 0-10); Function (as measured by the BASFI, 0-10); Inflammation (as measured by the mean of the two morning stiffness related questions from the BASDAI, 0-10); Spinal mobility (as measured by the BASMI, 0-10); Acute phase reactant (as measured by CRP)

ASAS Partial Remission Definition

A subject is defined as achieving partial remission if, and only if, they have a value of <2 units in each of the following 4 domains: Patient Global Assessment (measured on a VAS scale, 0-10); Pain (measured on a VAS scale, 0-10); Function (as measured by the BASFI, 0-10); Inflammation (as measured by the mean of the two morning stiffness related questions from the BASDAI, 0-10)

Bath Ankylosing Spondylitis Functional Index (BASFI)

The BASFI is a set of 10 questions designed to determine the degree of functional limitation in those patients with AS. The ten questions were chosen with a major input from patients with AS. The first 8 questions consider activities related to functional anatomy. The final 2 questions assess the patients' ability to cope with everyday life. A 10 cm visual analog scale is used to answer the questions. The mean of the ten scales gives the BASFI score—a value between 0 and 10.

Bath Ankylosing Spondylitis Disease Activity Index (BASDAI)

The BASDAI consists of a one through 10 scale (one being no problem and 10 being the worst problem), which is used to answer 6 questions pertaining to the 5 major symptoms of AS: 1. Fatigue; 2. Spinal pain; 3. Joint pain/swelling; 4. Areas of localized tenderness (called enthesitis, or inflammation of tendons and ligaments); 5. Morning stiffness duration; 6. Morning stiffness severity. To give each symptom equal weighting, the mean (average) of the two scores relating to morning stiffness is taken. The resulting 0 to 50 score is divided by 5 to give a final 0-10 BASDAI score. Scores of 4 or greater suggest suboptimal control of disease, and patients with scores of 4 or greater are usually good candidates for either a change in their medical therapy or for enrollment in clinical trials evaluating new drug therapies directed at Ankylosing Spondylitis.

Patient's Global Assessment of Disease Activity

The patient's global assessment of disease activity will be performed using a 100 mm VAS ranging from no disease activity to maximal disease activity, after the question “Considering all the wAS your arthritis affects you, draw a line on the scale for how well you are doing”. At the investigator's site the distance in mm from the left edge of the scale will be measured and the value will be entered on the eCRF.

Patient's Assessment of Pain Intensity

The patient's assessment of inflammatory back pain will be performed using a 100 mm VAS ranging from no pain to unbearable pain. At the investigator's site the distance in mm from the left edge of the scale will be measured and the value will be entered on the eCRF.

Bath Ankylosing Spondylitis Metrology Index (BASMI)

The BASMI is a validated instrument that uses the minimum number of clinically appropriate measurements that assess accurately axial status, with the goal to define clinically significant changes in spinal movement. Parameters include: 1. cervical rotation; 2. tragus to wall distance; 3. lumbar side flexion; 4. modified Schober's; 5. intermalleolar distance.

Maastricht Ankylosing Spondylitis Enthesis Score (MASES)

The Maastricht Ankylosing Spondylitis Enthesis Score (MASES) was developed from the Mander index, and includes assessments of 13 sites. Enthesitis sites included in the MASES index are: 1st costochondral, 7^{th} costochondral, posterior superior iliac spine, anterior superior iliac spine, iliac crest (all above will be assessed bilaterally), 5th lumbar spinous process, proximal Achilles (bilateral).

Leeds Enthesis Index (LEI)

LEI is a validated enthesis index that uses only 6 sites for evaluation of enthesis: lateral epicondyle humerus L+R, proximal achilles L+R and lateral condyle femur. While LEI demonstrated substantial to excellent agreement with other scores in the indication of psoriatic arthritis, LEI demonstrated a lower degree of agreement with MASES in ankylosing spondylitis and might thus yield additional information in this indication.

MRI

Magnetic resonance imaging (MRI) of the spine was performed using a scoring system for quantification of AS-related pathologies, to investigate whether these changes were affected by treatment with secukinumab. MRIs were acquired locally at the clinical sites, and images were transmitted, QC'd, de-identified (if necessary) and analyzed centrally (blinded review). MRI scans were collected at baseline (preferably within 2 weeks prior to first treatment) and at week 6 (±1 week) and week 28 (±1 week). MRI scans included pre- and post-intravenous gadolinium contrast enhanced MRI for evaluating inflammation and fat-saturating techniques such as short tau inversion recovery (STIR) to monitor bone marrow edema. The analysis method is the ‘Berlin modification of ASspiMRI-a’ (Lukas C et al (2007) J Rheumatol; 34(4):862-70 and Rudwaleit et al (2005) [abstract] Arthritis Rheum 50:S211), which scores inflammatory changes in nearly the entire vertebral column (C2-S1).

### Example 1.2

### Secukinumab Shows Good Safety and Efficacy in the Treatment of Active Ankylosing Spondylitis

Demographics and baseline characteristics were comparable between groups. Mean (SD) BASDAI at baseline was 7.1 (1.4) for secukinumab-treated patients and 7.2 (1.8) for placebo-treated patients. Three patients on placebo and 2 patients on secukinumab discontinued the study prior to the primary endpoint, mostly due to unsatisfactory therapeutic effect. Efficacy data from 1 patient was not available due to a protocol violation after randomization. At week 6, 14/23 secukinumab-treated patients who entered efficacy analysis achieved ASAS20 responses versus 1/6 placebo treated patients (61% vs 17%, probability of positive-treatment difference=99.8%, credible interval 11.5%, 56.3%) (Table 4).

TABLE 4
Week 6 results for trial CAIN457A2209

| | | | Difference (vs. | 95% credible | Probability |
|---|---|---|---|---|---|
| | # of Responders (%) | Response rate | placebo) | interval | (Drug > Pbo) |
| AIN457 | 14/23 (60.9%) | 59.2% | 34.7% | 11.5%, 56.3% | 99.8% |
| Placebo | 1/6 (16.7%) | 24.5% | | | |

ASAS40 and ASAS5/6 responses of secukinumab-treated patients were 30% and 35%, respectively, and mean (range) BASDAI change was −1.8 (−5.6 to 0.8). In a majority of the ASAS20 responders, secukinumab induced responses within a week of treatment. ASAS response rates were greatest at the primary endpoint at week 6, and declined thereafter up to end of study at week 28, consistent with the preliminary dose regimen of only two doses of 10 mg/kg given at days 1 and 22, as chosen for this proof-of concept study. Post-hoc analyses of subgroups showed superior response rates with TNF alpha antagonist naive patients (11/13; 85%) compared to TNF alpha antagonist pre-exposed patients (3/10; 30%). The pharmacokinetic profile was as expected for an IgG1 mAb and comparable to secukinumab given for other indications.

The primary endpoint of this study was met, as secukinumab induced significantly higher ASAS20 responses than placebo at week 6. No early safety signals were noted in this study population.

### Example 1.3

### Secukinumab Reduces Spinal Inflammation in Patients with AS as Early as Week 6, as Detected by Magnetic Resonance Imaging

Magnetic resonance imaging (MRI) is considered gold standard for assessment of spinal inflammation in AS. We thus determined whether clinical effects observed after 2 infusions (10 mg/kg i.v.) of secukinumab coincide with reductions of bone marrow edema seen on MRI. Sagittal MRI of the spine was performed including T1- and short tau inversion recovery (STIR) sequences at baseline (BL), week 6 and week 28. Images were analyzed by an independent reader, who was blinded to treatment allocation and chronology of images, using the “Berlin modification” of the AS spinal MRI (ASspiMRI-a) scoring system. Changes between baseline and follow-up in each treatment arm were evaluated by Wilcoxon signed-rank test.

Twenty seven patients (22 secukinumab; 5 on placebo) had evaluable MRI images at baseline. Few patients (at week 6: 2 secukinumab; 3 placebo; at week 28: 6 secukinumab, 1 placebo) missed follow-up MRIs, mostly due to early discontinuation. MRI scores at baseline and changes at week 6 and week 28 are shown in Table 5. MRI score improvements were seen as early as week 6 and sustained up to week 28. Early improvements at week 6 were especially noted in patients with higher baseline scores. Only minor changes were seen in patients on placebo.

TABLE 5
MRI scores and ASAS response at week 6 and 28 following treatment with secukinumab

| | Secukinumab 2 × 10 mg/kg | | | Placebo | | |
|---|---|---|---|---|---|---|
| | Baseline | Week 6 | Week 28* | Baseline | Week 6 | Week 28 |
| # of patients | 22 | 22 | 16 | 5 | 3 | 5 |
| ASAS20 | — | 14 | 6 | — | 1 | 1 |
| responders (n) | | | | | | |
| Mean Berlin | 9.2 ± 8.9 | 6.7 ± 6.6 | 5.7 ± 6.2 | 20.6 ± 20.2 | 21.0 ± 24.6 | 19.0 ± 19.3 |
| score ± SD | | | | | | |
| P-value (vs. | — | 0.10 | 0.16 | — | 0.50 | 0.25 |
| baseline) | | | | | | |

*Data from 6 patients who discontinued prior to week 28 (lack of response) were not analyzed.

The results of this exploratory study in patients with active AS suggests that after treatment with only 2 infusions of secukinumab, substantial reductions of spinal inflammation as detected by MRI occurred. MRI changes were seen as early as 6 weeks after start of treatment, and were maintained up to week 28. Results are consonant with MRI findings obtained in previous AS trials with TNF blockers. These results provide support that secukinumab may be a potential treatment for patients with active AS.

### Example 2

### Materials and Method for Pharmacogenetic Analysis in AS Trial CAIN457A2209

### Example 2.1

### Samples and Processing

DNA was genotyped in 27 consenting patients who participated in the study. 23 patients who received secukinumab were used in the pharmacogenetic (PG) analysis. One patient on secukinumab discontinued prior to week 6 and thus was excluded, resulting a PG analysis set of 22 patients.

Blood samples from consenting patients were collected at the individual trial sites and then shipped to Covance (Geneva, Switzerland). The genomic DNA of each patient was extracted from the blood by Covance using the PUREGENE D-50K DNA Isolation Kit (Gentra, Minneapolis, Minn., USA) and shipped to Novartis for genotyping.

A total of 11 SNPs reported to be associated with AS disease risk or IL17 pathway were genotyped. TaqMan® genotyping was performed using TaqMan Assays-by-Design and Assays-on-Demand (Applied Biosystems, Foster City, Calif.) on an ABI 7900HT sequence detection system. Up to 20 ng of genomic DNA was used in the experiment according to the manufacturer's instructions.

The HLA-B*27 allelic group was also chosen for genotyping given it is the major genetic risk factor for AS. In addition, the HLA-DRB1*04 allelic group (2-digit alleles) was included in the test because of the prior finding of association with differential response to secukinumab treatment in rheumatoid arthritis (RA) trials. All DNA samples from consenting patients in the study were tested with sequence-specific oligonucleotide hybridization (SSO) method. SSO experiments were performed by using LABType® HD B and DRB1 Typing Test (One lambda, Inc, CA) with Luminex IS200 instrument according to manufacturer's instructions. HLA genotypes were assigned by using HLA Fusion® 2.0 software (One Lambda).

### Example 2.2

### Statistical Analysis

Generally, statistical models for the pharmacogenetic analyses were based on the models used in the analysis of the clinical trials, adding a term for the genotype of the variant being tested. All variants were tested individually, i.e., only 1 variant was included in the model at a time. All SNPs were tested against clinical endpoints using the standard additive effect coding: individuals were coded 0, 1 or 2, depending on the number of copies of the less frequent allele that an individual carries. The allelic group HLA-B*27 was tested for association using the standard additive effect by coding individuals as 0, 1, or 2, depending on the number of copies of the HLA-B*27 allele that an individual carries. The allelic group HLA-DRB1*04 was tested for association using the standard additive effect by coding individuals as 0, 1, or 2, depending on the number of copies of the HLA-DRB1*04 allele that an individual carries

All association tests were two-tailed, single-point tests for an additive allelic effect. Only secukinumab-treated patients who continued to be in the study up to week 6 were used for the genetic analysis (N=22). The null hypothesis was that the coefficient for the genotype variable was equal to zero, and the corresponding p-value was presented. Rejecting the null hypothesis would mean concluding that genotype was a predictor of response to secukinumab as measured by the specific clinical endpoint.

All statistical tests were performed in SAS (SAS Institute Inc., Cary, N.C., USA). Efficacy variables ASAS20, ASAS40, ASAS5/6 at week 4/week 6 were analyzed separately using a logistic regression model (SAS 9.2 PROC GENMOD), and efficacy variable BASDAI at week 4/week 6 were analyzed separately using an ANCOVA model (SAS 9.2 PROC MIXED), with the efficacy endpoint as the dependent variable, SNP or HLA alleleic group genotype (as coded above) as the independent variable (fixed effect), and baseline BASDAI score as a fixed effect covariate. A permutation test was performed to account for small sample size and multiple comparisons of 13 genetic variants, with genotypes randomly permuted 250,000 times.

### Example 3

### Results for Pharmacogenetic Analysis in AS Trial CAIN457A2209

### Example 3.1

### Association of Genetic Variants with AIN457 Efficacy in Secukinumab-Treated Patients

A total of 13 variants including 11 SNPs and 2 HLA allelic groups were tested for association with efficacy endpoints (Table 6).

Among the 13 variants, a non-synonymous SNP of ERAP1 gene rs30187 has the best p value, with nominal p-value=8.14×10⁻⁵ for association with ASAS40 at week 6 (Table 7) and 4.45×10⁻³ for association with ASAS40 at week 4 (Table 8) in 22 patients using an additive model adjusting for baseline BASDAI score. Another SNP rs27434 of ERAP1 gene also showed nominally significant association with ASAS40 at week 6 (p-value=4.75×10⁻³).

A permutation test with 250,000 permutations on ASAS40 at week 6 returned a significant p-value (adjusted p-value=0.004) for rs30187, after accounting for the small sample size and multiple comparisons of 13 genetic variants.

The association of ERAP1 with AS disease was initially identified by Wellcome Trust Case Control Consortium (Burton et al. (2007) Nat. Genet., 39, 1329-1337), subsequently replicated and further confirmed in independent cohorts. ERAP1 gene is located on chromosome and encodes an aminopeptidase, which is an enzyme that cleaves other proteins into smaller fragments called peptides. ERAP1 protein has two major biological functions, both of which are important for normal immune system function. First, ERAP1 cleaves cytokine receptors on cell surface, which reduces their ability to transmit chemical signals into the cell and inturn affects the process of inflammation. Second, ERAP1 protein is involved in trimming peptides for major histocompatibility complex (MHC) presentation.

rs30187 is a non-synonymous (amino acid changing) SNP of ERAP1. It has previously been demonstrated that rs30187 causes a significant reduction in aminopeptidase activity toward a synthetic peptide substrate as well as alterations in substrate affinity (Goto et al (2008) Biochem. J., 416, 109-116). Strong correlation of ERAP1 expression in lymphoblastoid cell lines with SNPs close to and within ERAP1 has also been observed, including the marker rs30187 (Dixon et al. (2007) Nat. Genet., 39, 1202-1207).

| | | Alleles | |
|---|---|---|---|
| Variant | Gene | (minor/major) | Polymorphism position |
| rs11209032 | IL23R | A/G | downstream |
| rs2201841 | IL23R | G/A | intronic |
| rs11209026 | IL23R | A/G | non-synonymous |
| rs10865331 | — | A/G | genomic |
| rs2310173 | IL1R2 | A/C | downstream |
| rs4333130 | ANTXR2 | C/T | intronic |
| rs2242944 | — | A/G | genomic |
| rs27434 | ERAP1 | A/G | synonymous |
| rs30187 | ERAP1 | T/C | non-synonymous |
| rs1974226 | IL17A | T/C | 3′ UTR |
| rs7747909 | IL17A | A/G | 3′ UTR |
| HLA-DRB1*04 | HLA-DRB1 | | |
| HLA-B*27 | HLA-B | | |

Table 6 shows the gene, allele and position information of the 13 genetic variants tested.

| | | ASAS20 | ASAS40 | ASAS5/6 | BASDAI |
|---|---|---|---|---|---|
| | | week6 | week6 | week6 | week6 |
| Variant | Gene | p-value | p-value | p-value | p-value |
| rs11209032 | IL23R | 0.27 | 0.81 | 0.88 | 0.29 |
| rs2201841 | IL23R | 0.24 | 0.39 | 0.78 | 0.46 |
| rs11209026 | IL23R | 0.71 | 0.97 | 0.78 | 0.77 |
| rs10865331 | — | 0.64 | 0.75 | 0.16 | 0.24 |
| rs2310173 | IL1R2 | 0.18 | 0.46 | 0.22 | 0.70 |
| rs4333130 | ANTXR2 | 0.69 | 0.56 | 0.82 | 0.71 |
| rs2242944 | — | 0.12 | 0.34 | 0.71 | 0.28 |
| rs27434 | ERAP1 | 0.21 | 4.75E−03 | 0.042 | 0.47 |
| rs30187 | ERAP1 | 0.22 | 8.14E−05 | 0.022 | 0.22 |
| rs1974226 | IL17A | 0.56 | 0.87 | 0.26 | 0.64 |
| rs7747909 | IL17A | 0.40 | 0.36 | 0.53 | 0.68 |
| HLA-DRB1*04 | HLA-DRB1 | 0.25 | 0.62 | 0.70 | 0.49 |
| HLA-B*27 | HLA-B | 0.61 | 0.10 | 0.62 | 0.53 |

Table 7 shows the p-values from association tests for each genetic variant against ASAS20, ASAS40, ASAS5/6 and BASDAI at week 6.

| | | ASAS20 | ASAS40 | ASAS5/6 | BASDAI |
|---|---|---|---|---|---|
| | | week4 | week4 | week4 | week4 |
| Variant | Gene | p-value | p-value | p-value | p-value |
| rs11209032 | IL23R | 0.40 | 0.45 | 0.45 | 0.41 |
| rs2201841 | IL23R | 0.26 | 0.78 | 0.78 | 0.58 |
| rs11209026 | IL23R | 0.45 | 0.83 | 0.83 | 0.73 |
| rs10865331 | — | 0.89 | 0.49 | 0.96 | 0.86 |
| rs2310173 | IL1R2 | 0.29 | 0.59 | 0.59 | 0.75 |
| rs4333130 | ANTXR2 | 0.21 | 0.99 | 0.49 | 0.45 |
| rs2242944 | — | 0.89 | 0.30 | 0.69 | 0.41 |
| rs27434 | ERAP1 | 0.33 | 0.083 | 0.084 | 0.67 |
| rs30187 | ERAP1 | 0.029 | 4.45E−03 | 4.55E−03 | 0.26 |
| rs1974226 | IL17A | 0.20 | 0.90 | 0.90 | 0.23 |
| rs7747909 | IL17A | 0.60 | 0.20 | 0.67 | 0.24 |
| HLA-DRB1*04 | HLA-DRB1 | 0.74 | 0.65 | 0.65 | 0.36 |
| HLA-B*27 | HLA-B | 0.31 | 0.58 | 0.58 | 0.96 |

Table 8 shows the p-values from association tests for each genetic variant against ASAS20, ASAS40, ASAS5/6 and BASDAI at week 4.

### Example 3.2

### Effect of ERAP1 SNP rs30187 Alleles on Secukinumab Response in Secukinumab-Treated Patients

14 out of 22 secukinumab-treated patients have at least one rs30187 non-response allele (T). As shown in Table 9, a lower percentage of patients having at least one rs30187 AS non-response allele achieve ASAS20 and ASAS40 at both week 4 and 6 following treatment with secukinumab.

| Percent patients | | rs30187 | | |
|---|---|---|---|---|
| reaching endpoint | | CT or TT | rs30187 CC | All patients |
| in secukinumab arm | Week | (n = 14) | (n = 8) | (n = 22) |
| % reaching ASAS20 | week 4 | 35.7 | 75.0 | 50.0 |
| | week 6 | 57.1 | 75.0 | 63.6 |
| % reaching ASAS40 | week 4 | 21.4 | 62.5 | 36.4 |
| | week 6 | 7.1 | 75.0 | 31.8 |
| % reaching ASAS5/6 | week 4 | 21.4 | 62.5 | 36.4 |
| | week 6 | 28.6 | 50.0 | 36.4 |

Table 9 shows the percentage of secukinumab-treated patients reaching a given endpoint (ASAS20, ASAS40, ASAS5/6), grouped by the carrier/non-carrier status of rs30187 non-response allele.

The effect of rs30187 genotype on over time response to treatment with secukinumab may be seen in FIG. 2. Carriers of rs30187 non-response allele (those carrying one or two copies of T alleles) have a lower ASAS40 response rate than non-carriers (those not carrying T allele), consistently overtime up to week 10 since the first dosing of secukinumab.

### Example 3.3

### Effect of ERAP1 SNP rs27434 Alleles on Secukinumab Response in Secukinumab-Treated Patients

Another ERAP1 SNP tested in the study (rs27434; Australo-Anglo-American Spondyloarthritis Consortium (TASC) et al. (2010) Nat. Genet., 42(2):123-127) also showed nominally significant association with ASAS40 at week 6 (Table 7) but not week 4 (Table 8). 12 out of 22 secukinumab-treated patients have at least one rs27434 non-response allele (A). As shown in Table 10, a lower percentage of patients having at least one rs27434 non-response allele achieve ASAS20 and ASAS40 at both weeks 4 and 6 following treatment with secukinumab.

| Percent patients | | rs27434 | | |
|---|---|---|---|---|
| reaching endpoint | | AG or AA | rs27434 GG | All patients |
| in secukinumab arm | Week | (n = 12) | (n = 10) | (n = 22) |
| % reaching ASAS20 | week 4 | 41.7 | 60.0 | 50.0 |
| | week 6 | 58.3 | 70.0 | 63.6 |
| % reaching ASAS40 | week 4 | 25.0 | 50.0 | 36.4 |
| | week 6 | 8.3 | 60.0 | 31.8 |
| % reaching ASAS5/6 | week 4 | 25.0 | 50.0 | 36.4 |
| | week 6 | 25.0 | 50.0 | 36.4 |

Table 10 shows the percentage of secukinumab-treated patients reaching a given endpoint (ASAS20, ASAS40, ASAS5/6), grouped by the carrier/non-carrier status of rs27434 non-response allele.

The two ERAP1 SNPs rs30187 and rs27434 are 5,182 base pairs apart in the coding region of ERAP1 gene on chromosome 5 (human genome hg18 coordinates) and in moderate linkage disequilibrium (r²=0.63) according to 1000 Genomes pilot 1 CEU (Caucasian residents of European ancestry from Utah, USA) panel.

### Example 3.4

### Effect of ERAP1 SNPs rs30187 and rs27434 on Secukinumab Response in Secukinumab-Treated Patients Carrying HLA-B*27 Allele or not Previously Treated with Anti-TNF

A total of 41% of patients in the PG analysis set were previously treated with anti-TNF therapy. To test if patients with poor response to anti-TNF therapy (refractory) are refractory to treatment with secukinumab, analysis similar to that described in Example 3.1 was conducted by excluding the patients who were previously treated with anti-TNF therapy. As shown in Table 11, similar association between rs30187 and secukinumab response for ASAS40 at week 4 and week 6 was observed in the anti-TNF naïve subgroup as in all patients.

A total of 77% of patients in the PG analysis set carry at least one copy of HLA-B*27 allele. Interestingly, although HLA-B*27 is the main genetic risk factor for AS, our analysis did not identify a significant association between HLA-B*27 and efficacy endpoints (ASAS20, ASAS40, ASAS5/6 and BASDAI). It has been previously shown that ERAP1 SNPs only affect AS risk in HLA-B27-positive individuals (Evans et al. (2011) Nat Genet., 43(8):761-767). Thus, to test the association between ERAP1 SNPs and secukinumab efficacy in patients carrying HLA-B*27 allele, analysis similar to that described in Example 3.1 was conducted by excluding the patients without HLA-B*27 allele. As shown in Table 11, similar associations between rs30187 and secukinumab response for ASAS40 at both week 4 and 6, and for ASAS5/6 at week 4, were observed in the HLA-B*27 positive subgroup as in all patients.

| | | Anti-TNF naive pts (n = 13) | | | | HLA-B*27 positive pts (n = 17) | | | |
|---|---|---|---|---|---|---|---|---|---|
| | | ASAS20 | ASAS40 | ASAS5/6 | BASDAI | ASAS20 | ASAS40 | ASAS5/6 | BASDAI |
| SNP | Week | p-value | p-value | p-value | p-value | p-value | p-value | p-value | p-value |
| rs30187 | week 4 | 0.27 | 0.076 | 0.078 | 0.76 | 0.12 | 7.82E−03 | 8.07E−03 | 0.36 |
| | week 6 | 0.029 | 5.34E−03 | 0.21 | 0.64 | 0.49 | 2.22E−03 | 0.029 | 0.26 |
| rs27434 | week 4 | 0.74 | 0.30 | 0.30 | 0.82 | 0.51 | 0.08 | 0.080 | 0.75 |
| | week 6 | 0.029 | 0.038 | 0.19 | 0.80 | 0.18 | 0.023 | 0.027 | 0.33 |

Table 11 shows the p-values from association tests for ERAP1 SNPs against ASAS20, ASAS40, ASAS5/6 and BASDAI at week 4 and 6, in subgroups of secukinumab-treated patients carrying HLA-B*27 allele or not previously treated with anti-TNF.

### Example 3.5

### Effect of IL23R SNPs (rs11209032 and rs2201841) Alleles on Secukinumab Response in Secukinumab-Treated Patients

A similar analysis as that described in Example 3.1 was conducted for efficacy endpoints (ASAS20, ASAS40, ASAS5/6 and BASDAI) at week 28. Based on our analysis, we have determined that two SNPs of the IL23R gene (Safrany et al. (2009) Scandinavian Journal of Immunology 70, 68-74) have the best p-values, with nominal p-value=3.65×10⁻³ for association of SNP rs11209032 and p-value=8.52×10⁻³ for association of SNP rs2201841, with BASDAI at week 28.

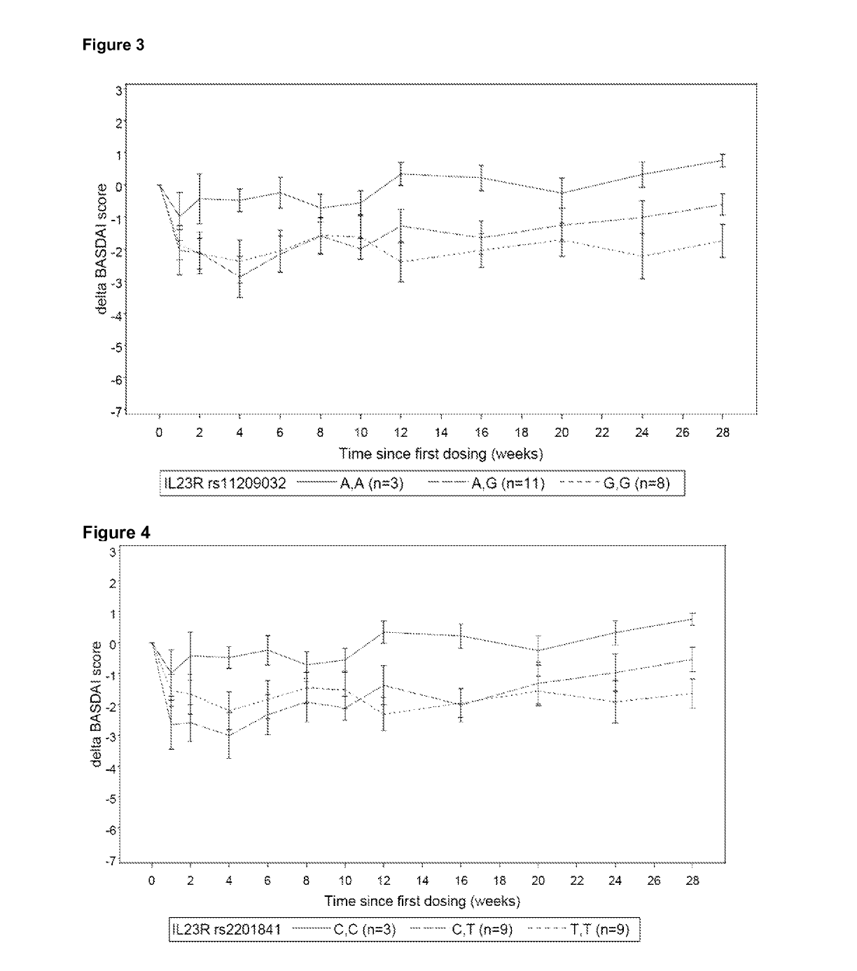

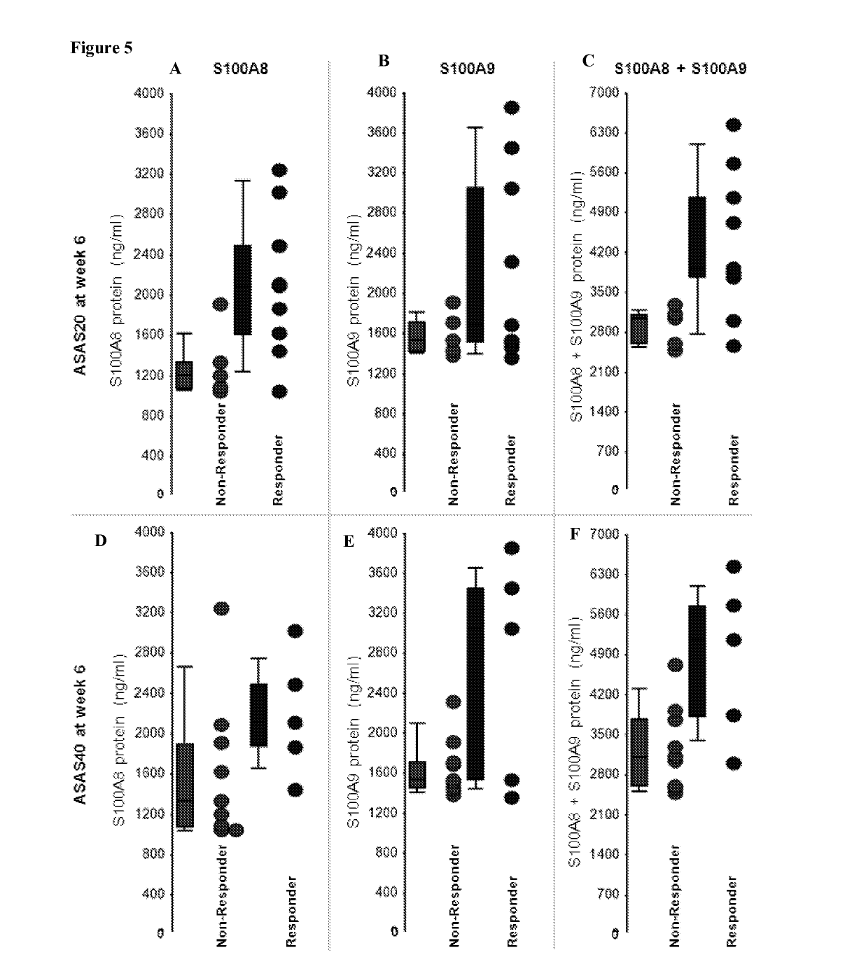

Three out of 22 secukinumab-treated patients have at least two rs11209032 AS non-response allele (A) and 11 secukinumab-treated patients have one rs11209032 AS non-response allele. As shown in FIG. 3, following secukinumab treatment, patients having at least one IL23R rs11209032 “G” allele display improved BASDAI scores over time relative to patients having two “A” alleles.

A similar effect was observed for IL23R rs2201841 (FIG. 4). Three out of 22 secukinumab-treated patients have at least two rs2201841 AS non-response allele (C) and nine secukinumab-treated patients have one rs2201841 AS non-response allele. Following secukinumab treatment, patients having at least one rs2201841 “T” allele display improved BASDAI scores over time relative to patients having two “C” alleles.

The two IL23R SNPs rs11209032 and rs2201841 are 45,890 base pairs apart in the IL23R gene on chromosome 1 (human genome hg18 coordinates) and in moderate linkage disequilibrium (r²=0.50) according to 1000 Genomes pilot 1 CEU (Caucasian residents of European ancestry from Utah, USA) panel. However, the two IL23R SNPs showed high linkage disequilibrium (r²=0.90) in this PG analysis set of 22 secukinumab-treated patients from AS Trial CAIN457A2209. The observed high linkage disequilibrium in this dataset indicates that the similar associations of rs11209032 and rs2201841 with BASDAI may be due to the strong correlation between the two SNPs and the observed phenotype (BASDAI) cannot be unequivocally assigned to a single SNP.

### Example 4

### Materials and Method for S100 Protein Analysis in AS Trial CAIN457A2209

### Example 4.1

### Sample Processing and Measurement of S100 Protein Levels

Plasma samples were obtained from subjects participating in AIN457A2209 at week 0 and week 6. Surrogate matrix and human serum samples (50 μL) were mixed with 250 μL, of 100 mM Tris buffer containing 1 mM CaCl₂, pH 8.0. The samples were reduced using commercially available reducing agents according to the manufacturer's recommendations. Trypsin (75 μL, 1 mg/mL) was added to each sample. The digestions were incubated overnight (16 hours) at 37° C. in a circulating water bath. The enzymatic reaction was terminated with 40 μL, of 10% formic acid in water (v/v). PDS (10 mM final concentration) was then added to each sample for 20 minutes. The samples were centrifuged at 5,000 rpm (2655 rcf) for 5 minutes. The S100A8, S100A9, S100A12 signature peptides and internal standards were identified by liquid chromatography coupled to tandem mass spectrometry (LC-MS/MS). Injections (50 μL) were made onto a 150×2.00 mm 4 micron Synergi Hydro-RP column (Phenomenex, Torrance, Calif., USA) using a CTC Analytics HTS Pal Autosampler (CTC Analytics, Switzerland) and a binary 1100 LC pump (Agilent, Santa Clara, Calif.). Mobile phase A was 0.1% formic acid in water. Mobile phase B was 0.1% formic acid in 90:10 acetonitrile/water (v/v). The total running time per sample was 20 minutes. An API-5000 triple quadrupole mass spectrometer (Applied Biosystems, Foster City, Calif.) was used for detection. LC-MS/MS data were acquired in MRM mode with positive electrospray ionization (ESI). Two transition ions (quantifier and qualifier) were monitored (dwell time: 200 milliseconds) for each signature peptide to confirm specificity. The elution times of the S100A8, S100A9, and S100A12 signature peptides were 14.6, 10.5, and 7.5 minutes, respectively.

The intensities of the S100A8, S100A9, and S100A12 signature peptides and internal standards were determined by integration of extracted ion peak areas using Analyst 1.5.1 software (Applied Biosystems, Foster City, Calif.). The signature peptide (quantifier) to internal standard peak area ratio was used for comparison across the samples. Calibration curves were prepared by plotting the signature peptide to internal standard peak area ratio vs. concentration (ng/mL). A calibration curve was established using seven concentrations. Data points were selected from calibrator-samples (C samples) at the beginning and end of the analytical run. The model for the calibration curve was linear with (1/×2) weighting consistent throughout the study. Calibration curves for S100A8, S100A9, and S100A12 signature peptides were prepared from freshly prepared C standards in surrogate matrix. Each calibration curve consisted of seven concentrations fit to a linear regression model with Analyst 1.5.1 software (Applied Biosystems, Foster City, Calif.). The correlation coefficients (r) of the three calibration curves were ≧0.95. The accuracies for 2/3 of the individual C standards were within ±15% (±20% for the LLOQ). The intra-assay (within day) accuracy and precision of the calibration curves, calculated as the mean bias (%) and precision (CV, %) of all individual concentrations of C standards analyzed that day, were within ±15%.

The accuracy and precision of the method was evaluated at three QC concentrations spiked in human serum. The endogenous levels of S100A8, S100A9, and S100A12 signature peptides were below the LLOQ of 5, 1 and 0.25 ng/mL, respectively.

### Example 5

### Results for S100 Protein Analysis in AS Trial CAIN457A2209

The baseline levels of a series of proteins were determined for secukinumab treated patients of the AIN457A2209 study: BDEF2, DKK1, IL17A, IL17F, IL22, MIP3A (CCL20), MMP3, NGAL, S100A8, S100A9 and S100A12. Protein levels were then compared between week 6 clinical responders and non-responders using either the ASAS20 or the ASAS40 cut-off to define response. None of the tested proteins, except S100A8 and S100A9, showed significantly different levels between responders and non-responders and were hence considered not informative for the prediction of response.

Group-wise comparison of S100 baseline protein levels between ASAS20 or ASAS40 responders and non-responders in the AIN457A2209 study, indicated significantly higher levels of S100A8 (2 sample t-test, p=0.041) and S100A8+S100A9 (p=0.031) proteins in the ASAS20 responders. S100A9 (p=0.026) and S100A8+S100A9 (p=0.017) proteins were significantly higher in the ASAS40 responders.

### Example 6

### Conclusion for PGx and S100 Protein Analysis in AS Trial CAIN457A2209

The presence of AS non-response allele ERAP1 rs30187 is observed to associate with worse response to secukinumab in samples from the AIN457A2209 study. A similar but less significant association was also observed for another ERAP1 SNP, rs27434. The data provided herein supports an association between these ERAP1 alleles and an AS patient's response to secukinumab. We have also shown that, following secukinumab treatment, patients having at least one IL23R rs11209032 “G” allele display improved BASDAI scores over time relative to patients having only the rs11209032 “A” allele, and that patients having at least one rs2201841 “T” allele display improved BASDAI scores over time relative to patients having only the rs2201841 “C” allele. These findings could not have been predicted based solely on the fact that that certain ERAP SNPs and IL23R SNPs are associated with an increased likelihood of a patient developing the AS disease. For example, as shown in Table 6, various other SNPs associated with AS disease did not predict AS patient response to IL-17 antagonism with secukinumab—including HLA-B*27, the main genetic risk factor for AS. As such, one cannot predict how a patient will respond to a drug based solely based on whether that patient carries an allele associated with a particular disease, such as AS. We have also shown that patients who had elevated baseline levels of S100A8, S100A9 or S100A8+S100A9 were more likely to show clinical improvement upon secukinumab treatment than patients with low baseline levels of these proteins, indicating that these protein measurements may be informative for decisions about the treatment strategy for AS patients. Adding the levels of S100A8 and S100A9 may compensate for some of the technical or biological variability and may provide more robust predictive information. We note that other proteins that are also related to the IL-17 pathway or to inflammatory signaling were not informative for the prediction of clinical benefit of treatment, indicating that predictive information is not a general feature of many IL-17- or inflammatory pathway members and could not itself be foreseen.

### Example 7

### ERAP1 Expression in AS Patients from CAIN457A2209

### Example 7.1

### Materials and Methods

ERAP1 gene transcript levels in whole blood from patients in study CAIN457A2209 were determined using Affymetrix DNA microarrays (Human Genome 133 Plus 2.0). In brief, patient blood samples were collected in PAXgene Blood RNA tubes according to the manufacturer's recommendations (PreAnalytiX, Hombrechtikon, Switzerland). Samples were stored at approximately −80° C. until the RNA extraction step. Total RNA was isolated with the PAXgene Blood RNA Kit (Qiagen, Hilden, Germany) according to the manufacturer's procedure. The total RNA was quantified by its absorbance at λ=260 nm (A_{260 nm}) and the purity was estimated from its A_{260 nm}/A_{280 nm} ratio. RNA integrity was assessed on a Caliper LabChip GX system. The RNA samples were stored at approximately −80° C. until the next step, cDNA synthesis. The synthesis of cDNA was carried out with a starting amount of approximately 50 ng total RNA using the NuGEN Ovation RNA Amplification System V2 including the Ribo-SPIA amplification process according to the instructions of the manufacturer (NuGEN Technologies Inc.; San Carlos, U.S.). The resulting cDNA from was hybridized to Affymetrix Human Genome 133 Plus 2.0 GeneChips as specified by the manufacturer. The arrays were scanned on a GeneChip Scanner 3000 7G, a GCOS (GeneChip Operating Software) controlled system. The scanned image was converted into numerical values of the probe intensity (Signal) using the Affymetrix Software and stored as “.cel”-file data. The “.cel” files were subjected to RMA (Robust Multichip Average) normalization (Rafael. A. Irizarry, Benjamin M. Bolstad, Francois Collin, Leslie M. Cope, Bridget Hobbs and Terence P. Speed (2003), Summaries of Affymetrix GeneChip probe level data Nucleic Acids Research 31(4):e15) using the standard Affymetrix “.cdf” file. Differential expression of the ERAP1 transcript (209788_s_at) was detected by comparing baseline transcript levels of week 6 ASAS40 responders and ASAS40 non-responders of the A1N457 treatment arm. Filtering cut-offs for this group comparison were: median fold change >=1.5×, p-value <=0.05 using a Wilcoxon test.

### Example 7.2

### Results

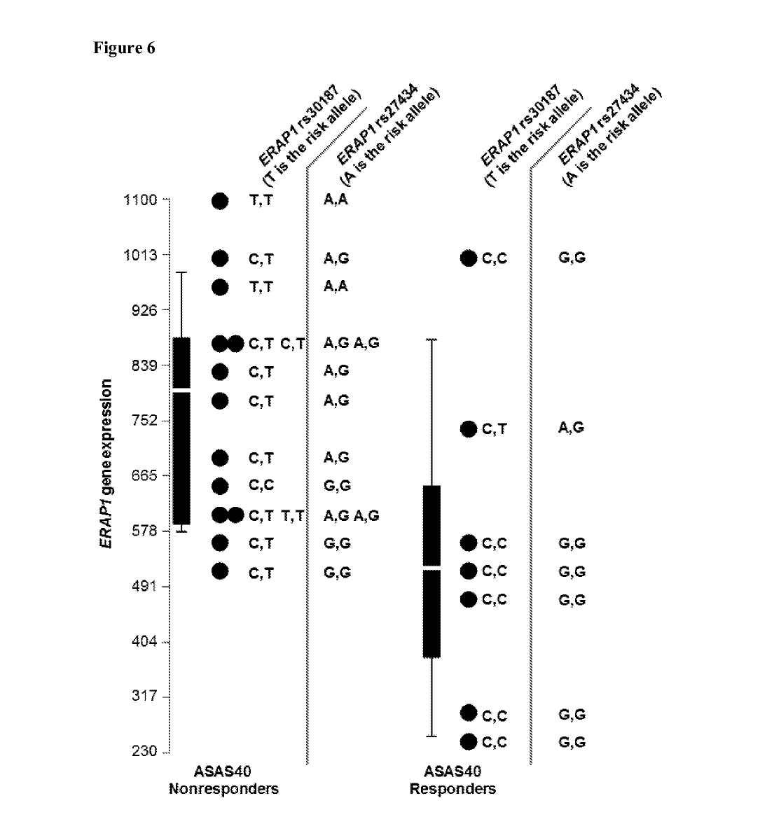

The results are presented in FIG. 6, which shows ERAP1 gene expression and genotype, along with the association with clinical response at week 6 in ASAS40 responders and nonresponders. Representations of ERAP1 gene expression levels and ERAP1 AS risk allele genotypes (rs30187 and rs27434) are combined. Dots represent individual secukinumab-treated patients at baseline. Week 6 ASAS40 nonresponders are represented on the left and responders on the right. Carriers of either of the ERAP1 risk alleles rs30187 (“T”) or rs27434 (“A”) typically showed higher levels of ERAP1 gene expression, while homozygous noncarriers mostly showed lower ERAP1 transcript levels. Additionally, a trend toward association with BASDAI changes over time was seen for IL-23R polymorphisms rs11209032 and rs2201841.

We show that carriers of either of the rs30187 “T” allele or rs27434 “A” allele typically showed higher levels of ERAP1 gene expression. It is known that the rs30187 protective allele “C” encodes an ERAP1 protein variant (K528R) with reduced catalytic activity relative to the ERAP1 protein encoded by the rs30187 risk allele “T” (Kochan et al. (2011) Proc Natl Acad Sci USA. 108(19):7745-50). Thus, it is reasonable to conclude that an increased level of ERAP1 expression and/or ERAP1 protein levels or activity may be useful to predict a diminished response to IL-17 antagonism (e.g., secukinumab) for AS patients, while a decreased level of ERAP1 expression and/or ERAP1 protein levels or activity may be useful to predict an improved response to IL-17 antagonism (e.g., secukinumab) for AS patients.

1.-50. (canceled)

51. A method of selectively treating a patient having ankylosing spondylitis (AS), comprising selectively administering a therapeutically effective amount of an IL-17 antagonist to the patient on the basis of said patient:
a) having at least one AS response allele selected from an rs2201841 response allele and an rs11209032 response allele; or
b) not having at least one AS non-response allele selected from an rs30187 non-response allele and an rs27434 non-response allele.

52. A method of selectively treating a patient having AS with an IL-17 antagonist, comprising:
a) selecting the patient for treatment with the IL-17 antagonist on the basis of the patient:
(i) having at least one AS response allele selected from an rs2201841 response allele and an rs11209032 response allele; or
(ii) not having at least one AS non-response allele selected from an rs30187 non-response allele and an rs27434 non-response allele; and
b) thereafter, administering a therapeutically effective amount of the IL-17 antagonist to the patient.

53. The method according to claim 52, further comprising, assaying a biological sample from the patient for the presence or absence of the at least one AS response allele or the at least one AS non-response allele, wherein the step of assaying is performed prior to selecting step a).

54. The method according to claim 53, wherein the presence or absence of the at least one AS non-response allele or the at least one AS response allele is detected by assaying the biological sample for a genomic sequence of the AS non-response allele or the AS response allele, a nucleic acid product of the AS non-response allele or the AS response allele, a polypeptide product of the AS non-response allele or the AS response allele, or an equivalent genetic marker of the AS non-response allele or the AS response allele.

55. The method according to claim 53, wherein
a) the biological sample is assayed for the presence or absence of an rs30187 non-response allele;
b) the biological sample is assayed for the presence or absence of an rs27434 non-response allele;
c) the biological sample is assayed for the presence or absence of an rs2201841 response allele; or
d) the biological sample is assayed for the presence or absence of an rs11209032 response allele.

56. The method according to claim 53, wherein the presence or absence of the at least one AS non-response allele or the at least one AS response allele in the biological sample is detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, immunoassays, immunohistochemistry, ELISA, flow cytometry, Western blot, HPLC, and mass spectrometry.

57. A method of predicting the likelihood that a patient having AS will respond to treatment with an IL-17 antagonist comprising, detecting:
a) the presence of at least one AS non-response allele selected from an rs30187 non-response allele and an rs27434 non-response allele; or
b) the presence of at least one AS response allele selected from an rs2201841 response allele and an rs11209032 response allele,
in a biological sample from the patient, wherein the presence of the at least one AS non-response allele is indicative of a decreased likelihood that the patient will respond to treatment with the IL-17 antagonist; and the presence of the at least one AS response allele is indicative of an increased likelihood that the patient will respond to treatment with the IL-17 antagonist.

58. A method for producing a transmittable form of information for predicting the responsiveness of a patient having AS to treatment with an IL-17 antagonist, comprising:
a) determining a decreased likelihood that the patient will respond to treatment with the IL-17 antagonist based on the presence of at least one AS non-response allele in a biological sample from the patient, wherein the at least one AS non-response allele is selected from an rs30187 non-response allele and an rs27434 non-response allele; or determining an increased likelihood that the patient will respond to treatment with the IL-17 antagonist based on the presence of at least one AS response allele in a biological sample from the patient, wherein the at least one AS response allele is selected from an rs2201841 response allele and an rs11209032 response allele; and
b) recording the result of the determining step on a tangible or intangible media form for use in transmission.

59. The method according to claim 51, wherein the patient has not been previously treated for AS, is TNF alpha antagonist naive, is a TNF-IR or is a TNF non-responder.

60. The method according to claim 53, wherein the biological sample is selected from the group consisting of synovial fluid, blood, serum, feces, plasma, urine, tear, saliva, cerebrospinal fluid, a leukocyte sample and a tissue sample.

61. The method according to claim 51, wherein the step of administering comprises either:
i) intravenously administering three doses of about 10 mg/kg of the IL-17 antagonist to the patient every other week, and thereafter subcutaneously administering about 75 mg-about 300 mg of the IL-17 antagonist to the patient monthly, beginning one month from delivery of the third intravenous dose; or
ii) subcutaneously administering five doses of about 75 mg-about 300 mg of the IL-17 antagonist to the patient weekly, and thereafter subcutaneously administering about 75 mg-about 300 mg of the IL-17 antagonist to the patient monthly, beginning one month from delivery of the fifth subcutaneous dose.

62. The method according to claim 51, wherein the step of administering comprises either:
i) intravenously administering about 10 mg/kg of the IL-17 antagonist to the patient during week 0, 2, and 4, and thereafter subcutaneously administering about 75 mg-about 300 mg of the IL-17 antagonist to the patient monthly, beginning during week 8; or
ii) subcutaneously administering about 75 mg-about 300 mg of the IL-17 antagonist to the patient during week 0, 1, 2, 3, and 4, and thereafter subcutaneously administering about 75 mg-about 300 mg of the IL-17 antagonist to the patient monthly, beginning during week 8.

63. The method according to claim 51, wherein the IL-17 antagonist is selected from the group consisting of:
a) an IL-17 antibody that binds to an epitope of IL-17 comprising Leu74, Tyr85, His86, Met87, Asn88, Val124, Thr125, Pro126, Ile127, Val128, His129;
b) an IL-17 antibody that binds to an epitope of IL-17 comprising Tyr43, Tyr44, Arg46, Ala79, Asp80;
c) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain;
d) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 binding molecule has a K_{D} of about 100-200 pM, and wherein the IL-17 binding molecule has an in vivo half-life of about 23 to about 35 days; and
e) an IL-17 antibody comprising:
i) an immunoglobulin heavy chain variable domain (V_{H}) comprising the amino acid sequence set forth as SEQ ID NO:8;
ii) an immunoglobulin light chain variable domain (V_{L}) comprising the amino acid sequence set forth as SEQ ID NO:10;
iii) an immunoglobulin V_{H} domain comprising the amino acid sequence set forth as SEQ ID NO:8 and an immunoglobulin V_{L} domain comprising the amino acid sequence set forth as SEQ ID NO:10;
iv) an immunoglobulin V_{H} domain comprising the hypervariable regions set forth as SEQ ID NO:1, SEQ ID NO:2, and SEQ ID NO:3;
v) an immunoglobulin V_{L} domain comprising the hypervariable regions set forth as SEQ ID NO:4, SEQ ID NO:5 and SEQ ID NO:6;
vi) an immunoglobulin V_{H} domain comprising the hypervariable regions set forth as SEQ ID NO:11, SEQ ID NO:12 and SEQ ID NO:13;
vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

64. The method according to claim 63, wherein the IL-17 antagonist is secukinumab.

65. A method of selectively treating a patient having AS, comprising selectively administering a therapeutically effective amount of an IL-17 antagonist to the patient on the basis of said patient having a decreased level of ERAP1 expression, ERAP1 protein or ERAP1 activity relative to a control.

66. A method of selectively treating a patient having AS, comprising selectively administering a therapeutically effective amount of an IL-17 antagonist to the patient on the basis of said patient having a test level of at least one AS response protein selected from the group consisting of S100A8, S100A9 and S100A8+S100A9 that is greater than a control level of the least one AS response protein.


SEQUENCE LISTING (17 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - DNA, 711 bp, homo sapiens
  CDS (1)..(708)
accatggaaaccccagcggagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 15 - protein, 236 aa, homo sapiens
TMETPAELLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 783 bp, homo sapiens
  CDS (1)..(780)
accatggaattggggctgagctgggttttccttgttgctattttagaaggtgtccactgtgaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccataa

SEQ ID NO: 17 - protein, 260 aa, homo sapiens
TMELGLSWVFLVAILEGVHCEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP